(19) United States
(12) Patent Application Publication
(10) Pub. No.: US 2023/0174571 A1
(43) Pub. Date: Jun. 8, 2023

(54) METHOD OF TREATING SHIP1-MEDIATED DISEASES USING PELOROL DERIVATIVES

(71) Applicant: Zebrapeutics Inc., Toronto (CA)

(72) Inventors: Xiao-Yan Wen, Toronto (CA); Raymond John Andersen, Vancouver (CA); Alice Low-Fung Mui, Burnaby (CA); Yuanyuan Zhao, Guang-dong (CN)

(21) Appl. No.: 17/920,169

(22) PCT Filed: Apr. 20, 2021
(86) PCT No.: PCT/CN2021/088448

§ 371 Date: Oct. 20, 2022

Related U.S. Application Data
(60) Provisional application No. 63/012,605, filed on Apr. 20, 2020.

(51) Int. Cl. C07J 69/00; C07J 71/00; A61P 29/00; A61P 1/16; A61P 1/04; A61P 37/08; A61P 35/00

(52) U.S. Cl. CPC C07J 69/00; C07J 71/0068; C07J 71/0052; A61P 29/00; A61P 1/16; A61P 1/04; A61P 37/08; A61P 35/00

(57) ABSTRACT

Provided are compounds of Formula I and pharmaceutically acceptable salt, solvate and/or derivative thereof. Further, provided are methods of treating a disease, disorder or condition mediated or treatable by activation of SHIP1 comprising administering a compound of Formula I or a pharmaceutically acceptable salt, solvate or derivative thereof. The compound of Formula I or a pharmaceutically acceptable salt, solvate or derivative thereof may be used in the treatment of SHIP1 mediated disease, disorder or conditions including inflammatory bowel disease (IBD), Crohn' disease, ulcerative colitis, multiple myeloma, liver injury, acute hepatitis and severe sepsis.

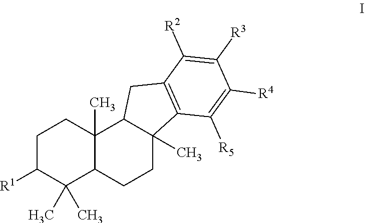

### FIELD

The present disclosure relates to compounds of and their use in the treatment of SHIP1-mediated disease, disorder or conditions such as inflammatory bowel disease (IBD), Crohn's disease, ulcerative colitis, multiple myeloma, liver injury, acute hepatitis and severe sepsis.

### INTRODUCTION

Src homology 2-containing inositol 5′-phosphatase 1 (SHIP1) and SHIP2 are both important phosphatases in mammalian cells that control various cellular signaling pathways including the PI3K/Akt pathway and production of interleukin-10 (IL-10). These cell signaling pathways are involved in a number of diseases and conditions mediated by SHIP1.

Multiple myeloma (MM), the second most common hematological malignancy (Naymagon 2016), is a plasma cell neoplasm characterized by an increase in malignant B cells and associated monoclonal immunoglobulin proteins in the bone marrow (BM) (Kuehl 2002). Despite current treatment options, including high-dose chemotherapy and stem-cell transplantation, the vast majority of patients experience a relapse of the disease, which remains incurable due to the development of drug resistance (Naymagon 2016, Abramson 2018, Harding 2019).

Cell-cell and cytokine-mediated interactions between MM cells and the BM microenvironment support proliferation, survival and drug resistance through activation of many signaling cascades including the Ras/Raf/Erk, Jak2/STAT3 and the PI3K/Akt pathways (reviewed in Harding 2019), and therefore numerous potential targets exist for therapeutic intervention. Signaling through the PI3K/Akt cascade is important for survival and expansion of neoplastic plasma cell clones and development of drug resistance (Hu 2018, Zhu 2015, Hideshima 2001, Qiang 2002, Tu 2000, Hsu 2001, Mitsiades 2002). Activation of PI3K leads to the production of phosphatidylinositol-3,4,5-trisphosphate (PIP₃) in the plasma membrane, which leads to membrane recruitment and activation of Akt and other pleckstrin homology (PH) domain-containing proteins (Zhu 2015). In MM, the level of phosphorylation of the serine/threonine kinase, Akt, and its downstream effectors correlates with the progression of the disease (Hsu 2001, Alkan 2002), and inhibitors of Akt and the downstream mammalian target of rapamycin (mTOR), have been shown to induce apoptosis in vitro and in vivo (Hideshima 2006, Frost 2004, Hideshima 2007). Thus, inhibiting PI3K/Akt signaling is a promising approach for MM therapy.

Cellular PIP₃ levels are tightly controlled under normal conditions by regulating both the activity of PI3K which generates PIP3 and the inositol lipid phosphatases that hydrolyze PIP₃. There are two main phosphatases that degrade PIP₃: the 3′-phosphatase PTEN, which produces PI-4,5-P₂, and the 5′-phosphatases SHIP1 and SHIP2, which produce PI-3,4-P₂ (Vivanco 2002). PTEN and SHIP2 are expressed in all cells, while SHIP1 is expressed only in hematopoietic cells. PTEN is a known tumor suppressor (Steck 1997, Li 1997) and PTEN deficient MM cells have higher Akt phosphorylation and are more sensitive to killing by Akt inhibition (Ge 2000, Shi 2002, Zhang 2003). SHIP1, on the other hand, is an important regulator of PI3K signaling in B cells (Aman 1998, Liu 1999, Helgason 2000), and reduced activity or expression has been observed in hematological malignancies (Luo 2004, Fukuda 2005, Vanderwinden 2006, Liang 2006). Agents currently being developed to reverse elevated PI3K/Akt signaling include kinase inhibitors targeting PI3K, Akt, or mTOR (Naymagon 2016, Abramson 2018, Harding 2019, Hu 2003, Zhu 2014). Activation of SHIP1 presents a distinct approach that could be used alone or complementary to existing therapies. (Li 2011, Meimetis 2012, Ong 2007) It has been shown in vitro that compounds in the Pelorol family selectively activate SHIP1 phosphatase activity by binding to an allosteric activation domain within the enzyme (Ong 2007). These compounds inhibit PI3K/Akt signaling in vitro within MM but not within non-hematopoietic cancer cells and this is associated with decreased proliferation and increased apoptosis of MM cells (Kennah 2009).

Inflammatory bowel disease (IBD) is another example where SHIP1 contributes to underlying mechanism of the disorder. Many factors contribute to the development of IBD, but genome wide association studies (Verstockt 2018) and clinical data (Engelhardt 2014, Clocker 2009, Clocker 2011, Louis 2009) show the anti-inflammatory actions of the cytokine interleukin-10 (IL10) (Ouyang 2011) is key in maintaining proper immune homeostasis. In its absence, stimulatory pathways proceed unabated leading to inappropriate inflammation. IL10 deficient mice develop colitis similar to human IBD (Kuhn 1993, Shouval 2014). In humans, polymorphisms in the IL10 gene are associated with ulcerative colitis and homozygous loss-of-function mutations in the IL10 receptor subunits result in early onset colitis (Engelhardt 2014, Clocker 2009, Clocker 2011).

It has been shown that activation of SHIP1 is required for IL10 to inhibit inflammation (Chan 2012, Cheung 2013). SHIP1 is a cytoplasmic protein expressed predominantly in hematopoietic cells (Fernandes 2013, Huber 1999, Krystal 2000). In response to extracellular signals, SHIP1 can be recruited to the cell membrane and one of its actions is to turn off phosphoinositide 3-kinase (PI3K) signaling (Brown 2010) by dephosphorylating the PI3K product PIP₃ into PI-3,4-P₂ (Fernandes 2013, Huber 1999, Krystal 2000, Pauls 2017). SHIP1 can also act as a docking protein for assembly of signaling complexes (Pauls 2017). It has been shown that SHIP1 is an allosterically regulated enzyme and its natural agonist is its product PI-3,4-P₂ (Ong 2007). Compounds of the Pelorol family are able to bind SHIP1's allosteric domain to activate SHIP1 (Ong 2007). In vitro results suggest that compounds of the Pelorol family exhibits anti-inflammatory effect in a manner similar to IL-10 (Chan 2012, Cheung 2013, Ong 2007).

Activation of anti-inflammatory pathways such as IL-10 can be useful in other situations. One example of heightened inflammatory response is sepsis. Sepsis is a complex systemic disease in which a dysregulated inflammatory response to bacterial or viral infection leads to the development of multi-organ dysfunction syndrome (MODS). The worldwide incidence is estimated to be 31 million cases per year. Severe sepsis accounts for 2% of patients admitted to the hospital and 10% of all intensive care unit admissions. Severe sepsis strikes young and old alike with an estimated mortality rate of 38% to 45%. In past decades, over 100 clinical trials of drugs for severe sepsis have failed, underlining the complexity and difficulty in treating of this disease, and the current therapy for this devastating syndrome is primarily supportive. (Marshall 2014) The currently pandemic COVID-19 is one type of viral sepsis. Mortality of COVID-19 patients is dominantly due to Acute Respiratory Distress Syndrome (ARDS). ARDS arises from a dysregulated host immune response to viral or bacterial infection, which is the hallmark of severe sepsis. This host over-response causes a “cytokine storm” (Liu 2016), resulting in systemic capillary vascular leakage, severe lung edema, ARDS, and patient death.

Inflammation is also commonly observed in various liver diseases such as viral hepatitis, autoimmune hepatitis, primary biliary cirrhosis, and liver allograft rejection, which are associated with activation and infiltration of T cells, production of pro-inflammatory cytokines in the liver, resulting in liver injury. (Louis 2003, Asdullah 2003, Czaja 2021)

Therefore, SHIP1 presents a viable target to develop a therapy that targets inflammatory diseases and neoplastic disorders. There exists a need to develop small molecule SHIP1 agonists for the treatment of such diseases.

### SUMMARY

It has been shown presently that compounds of the present disclosure can activate SHIP1 both in vitro and in vivo and are useful in the treatment of SHIP1-mediated conditions described below. Further, it has been shown that compounds of the present disclosure can reduce the level of tumour necrosis factor α (TNFα) in LPS-induced cells, and reduce the level of pro-inflammatory cytokines in colitis mouse models. Additionally, it has been shown that the compounds of the present disclosure reduce tumour mass in vivo in animals bearing MM tumours. Further, it has been shown presently that the compounds of the present disclosure inhibit inflammation in vivo in mouse IL-10 knockout models of IBD. Further, it has been shown that the compounds of the present disclosure protected concanavalin A (ConA) induced liver injury in mice. Moreover, treatment with the compounds of the present application increased the survival rate in septic mice in a Cecal Ligation and Puncture (CLP) model.

Since SHIP1 activation leads to stimulation of anti-inflammatory IL-10 signaling pathway, activation of the SHIP1 can be useful in inhibiting cytokine production through the IL10 pathway in a variety of diseases and conditions caused by inflammation. Examples include the cytokine storm observed in sepsis and over production of cytokines in various liver injuries.

Accordingly, the present disclosure includes a compound for Formula I

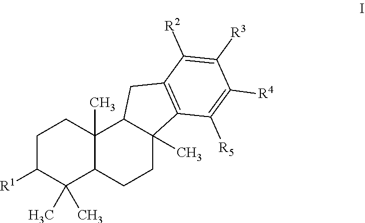

or a pharmaceutically acceptable salt, solvate, prodrug and/or derivative thereof,
wherein R¹ is selected from H, OH, C₁₋₃alkyl, OC₁₋₃alkyl, NH₂, NHC₁₋₃alkyl, NHSO₂C₁₋₃alkyl, NSuccinamide, and NHC(O)C₁₋₃alkyl;
wherein R², R³, R⁴, and R⁵ are independently selected from H, OH, OC₁₋₃alkyl, NH₂, NHC₁₋₃alkyl, NHSO₂C₁₋₃alkyl, and NHC(O)C₁₋₃alkyl; or R² and R³, R³ and R⁴ or R⁴ and R⁵ taken together with the atoms they are attached to form a substituted or unsubstituted 5- or 6-membered heterocycle comprising at least one NH and optionally one or more additional heteroatoms selected from N, O, and S; and
wherein when R² and R³, R³ and R⁴ or R⁴ and R⁵ are taken together to form the substituted or unsubstituted 5- or 6-membered heterocycle, R⁴ and R⁵, R² and R⁵, or R² and R³ respectively are independently selected from H and C₁₋₃alkyl.

In another aspect, the present disclosure includes a method of treating a disease, disorder or condition mediated or treatable by activation of SHIP1 comprising administering a therapeutically effective amount of a compound of the present disclosure or a pharmaceutically acceptable salt, solvate, prodrug and/or derivative thereof in a subject in need thereof.

In another aspect, the present disclosure includes a compound of the present disclosure or a pharmaceutically acceptable salt, solvate, prodrug and/or derivative for use in the treatment of a disease, disorder or condition mediated or treatable by activation of SHIP1.

In another aspect, the present disclosure includes a use of one or more compounds of the present disclosure or a pharmaceutically acceptable salt, solvate, prodrug and/or derivative thereof in the treatment of a disease, disorder or condition mediated or treatable by activation of SHIP1.

In another aspect, the present disclosure includes a use of one or more compounds of the present disclosure or a pharmaceutically acceptable salt, solvate, prodrug and/or derivative thereof in the manufacture of a medicament for the treatment of a disease, disorder or condition mediated or treatable by activation of SHIP1.

### DRAWINGS

The embodiments of the disclosure will now be described in greater detail with reference to the attached drawings in which:

FIG. 1 shows serum TNFα level of SHIP1^{+/+} or SHIP1^{-/-} mice injected intra-peritoneally with LPS, LPS+IL10 (Panel A), or LPS+compound I-1 (ZPR-100, or ZPR-MN100, or MN-100) (Panel B) at the concentrations indicated for 1 h. Data represent means of *p<0.05, **p<0.01 when compared with LPS-alone-stimulated mice, ns=not significant. Panel C shows STAT3^{+/+}, STAT^{-/-}, SHIP1^{+/+}, and SHIP1^{-/-} bone marrow-derived macrophages (BMDM) were stimulated with LPS (dotted line) or LPS+IL10 (solid line) over the course of 180 min in a continuous-flow apparatus. Fractions were collected every 5 min for measurement of TNFα levels. Data are representative of two independent experiments.

FIG. 2 shows IL-10 induces physical association of SHIP1 and STAT3. In Panel A, J17 SHIP1^{-/-} cells expressing either His₆-SHIP1 or His₆-SHIP1 3PT were tested for their ability to be inhibited by IL10 in a LPS-stimulated TNFα production assay. In Panel B, J17 His₆-SHIP1 cells were stimulated with IL6, 11_10 or Compound I-2 for 5 minutes. His₆-SHIP1 was pulled down using Nickel beads and along with cell lysates probed with SHIP1, STAT3 and phospho-STAT3 antibodies. (Panel C) Single cell FRET analysis of J17 SHIP1^{-/-} cells expressing FRET pair fusion constructs, Clover-SHIP1 and mRuby2-STAT3, ‘mock’ stimulated or stimulated with IL6, IL10, compound I-2 for 1 minute. FRET efficiency was determined using the Acceptor Photobleaching method. Data represent % FRET efficiency of single cells from at least three independent experiments for each treatment (One-Way ANOVA with Tukey's correction, ****p<0.0001).

FIG. 3 shows SHIP1 Y190 is involved in SHIP1 and STAT3 complex formation (A) TNFα production of 1 ng/ml LPS+IL10 stimulated SHIP1 KO cells reconstituted with WT or mutant SHIP1 or vector (none) determined by ELISA from which 1050 values for IL10 were calculated (One-Way ANOVA with Dunnett's correction ****p<0.0001). (B) Cells expressing either WT or Y190F SHIP1 were stimulated with IL10 or Compound I-2 for 5 minutes. His₆-SHIP1 was pulled down using Nickel beads and along with cell lysates probed with SHIP1, STAT3 and phospho-STAT3 and actin antibodies. (C) The amount of STAT3 protein being pulled down with His₆-SHIP1 (WT or Y190F) were quantified (Two-Way ANOVA with Sidak's correction, **p<0.01, *p<0.05).

FIG. 4 shows IL10 induces nuclear translocation of SHIP1 and STAT3. (A) SHIP1+/+, and STAT3+/+perimacs were stimulated with IL10 or compound I-2 for 2 or 20 minutes and stained with CD11b, SHIP1 and STAT3 antibodies and DAPI as indicated. (B) Pearson's coefficients were calculated to show the degree of overlap of SHIP1 or STAT3 with the membrane marker CD11b or DNA marker DAPI. Data represent Pearson's coefficients for individual fields of cells from at least two independent experiments in each cell type (Two-Way ANOVA with Sidak's correction, ****p<0.0001, ***p<0.001, **p<0.01, *p<0.05).

FIG. 5 shows PPAC, PAC1 and PAC2 have similar enzymatic activity as full length SHIP1. (A) Schematic diagram of the different SHIP1 truncation constructs. PPAC consists of PH-R domain, phosphatase and C2 domain (residues aa 293-877) PAC1 and PAC2 consists of phosphatase and C2 domain (residues aa 402-861 and aa 402-857 respectively). PAC1-cc and PAC2-cc contain surface entropy reduction mutations in C2 domain (E770A, E772A, E773A). This cluster of residues were identified using the SERp server (http://services.mbi.ucla.edu/SER/intro.php). (B) Enzyme catalytic initial velocities were determined at the indicated concentrations of IP4. Kcat and Km values were calculated using GraphPad software (C) Ability of compound I-1 to stimulate phosphatase activity in full length SHIP1, PPAC and PAC (Two-Way ANOVA with Tukey correction for multiple comparisons, **p<0.01, ****p<0.0001).

FIG. 6 shows PAC2 wild-type and mutant proteins response to compound I-2 and PI(3,4)P₂ and TNFα level of cells expressing wild-type or mutant SHIP1 or no SHIP1. (A) Bio-layer interferometry (BLI) data of PAC2 WT and K681A loaded sensors exposed to either 20 μM of compound I-2 or PI(3,4)P₂. ****p<0.0001 comparing WT PAC2 and K681A (Unpaired Student's t-test) (B) TNFα production of 10 ng/ml LPS+IL10 stimulated cells reconstituted with WT or K681A SHIP1 or none (SHIP1 KO) determined by ELISA from which IC50 values for IL10 were calculated. ****p<0.0001 when comparing to cells reconstituted with WT SHIP1 (Unpaired Student's t-test).

FIG. 7 shows AQX-1125/Rosiptor binding to PAC2 is weak compared to compound I-2 and PI(3,4)P2 (A) Structures of compound I-1 and its derivative compound I-2, and AQX-1125/Rosiptor (B) Representative Bio-layer interferometry (BLI) curves for binding of 20 μM compound I-2, AQX-1125 and PI(3,4)P2 to wild-type (WT) PAC2. Each data point indicates data from an independent biosensor (One-Way ANOVA with Tukey's correction ***p<0.001, **p<0.01).

FIG. 8 shows compounds I-1's effect on inflammation in IL10 colitis. (A) Representative H&E stained proximal, mid, and distal colon sections and pathological scores (B) of normal (no colitis, n=6) and colitic IL10 mice treated with vehicle (Veh., n=9), I-1 (3 mg/kg) (n=8) or dexamethasone (Dex., 0.4 mg/kg) (n=3) for 3 weeks. ****p<0.0001 when comparing to vehicle treated group (One-way ANOVA with Tukey's correction, F=34.59). (C) RT-qPCR of cDNA prepared from colonic sections of normal (No Colitis) and colitic IL10 mice treated with vehicle (Veh.), I-1 (3 mg/kg), or Dexamethasone (Dex., 0.4 mg/kg). Data represent mean IL-17 and CCL2 expression relative to GAPDH. **p<0.01, ****p<0.0001 when comparing to vehicle treated group (One-way ANOVA with Tukey's correction, F=34.59)

FIG. 9 shows IL10 and IL6 stimulates phosphorylation of STAT1 and STAT3 in BMDM. Cells were stimulated with 10 or 100 ng/mL IL10/IL6 for 30 minutes and lysates prepared for Immunoblot analysis with antibodies to the indicated proteins and phosphoproteins.

FIG. 10 shows compound I-1 inhibits MM cell growth in vivo. MM.1S cells expressing firefly luciferase were injected along with Matrigel basement membrane into the upper flank of NOD/SCID mice and allowed to establish for 2 weeks. Compound I-1 or control vehicle (n=4) were administered subcutaneously in an oil depot in the lower flank at a dose of 50 mg/kg of body weight every 3 days. (A) Bioluminescence images of control and compound I-1 treated mice. (B) Tumor volume was quantified using bioluminescence imaging.

FIG. 11 shows the effect of compound I-1 in the protection of ConA-induced liver injury. Panels A to D show plasma enzyme levels of alanine aminotransferase (ALT) and aspartate aminotransferase (AST), and levels of total bilirubin (TBIL) and blood urea nitrogen (BUN) respectively of C57 mice treated with blank control, compound I-1 (MN-100) alone, ConA alone, ConA with compound I-1 (MN-100) (3 mg/kg/d) or ConA with compound I-1 (MN-100) (10 mg/kg/d). Panels E and F show plasma enzyme levels of ALT and AST respectively of C57 mice treated with blank control, ConA alone, ConA with compound I-1 (MN-100) (10 mg/kg/d), or ConA with dexamethasone (0.5 mg/kg/d) (positive control).

FIG. 12 shows pictures of agar plates of blood culture from blank/vehicle control mice (Panel A), Caecum Ligation and Puncture (CLP)-operated septic mice (Panel B), or sham control mice (Panel C).

FIG. 13 shows dose-dependent therapeutic effect of compound I-1 (ZPR-MN100) in the treatment of CLP-septic mice. CLP operated mice were treated with 3 mg/kg/day or 10 mg/kg/day of I-1 by oral gavage and were compared to controls for survival rate.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DESCRIPTION OF VARIOUS EMBODIMENTS

### I. Definitions

Unless otherwise indicated, the definitions and embodiments described in this and other sections are intended to be applicable to all embodiments and aspects of the present disclosure herein described for which they are suitable as would be understood by a person skilled in the art.

The term “compound(s) of the disclosure” or “compound(s) of the present disclosure” and the like as used herein refers to a compound of Formula I or pharmaceutically acceptable salts, solvates, prodrug and/or derivatives thereof.

The term “composition(s) of the disclosure” or “composition(s) of the present disclosure” and the like as used herein refers to a composition, such a pharmaceutical composition, comprising one or more compounds of the disclosure.

The term “ZPR-100”, “ZPR-MN100”, “AQX-MN100” or “MN-100” as used here in refers to the compound I-1.

The term “ZPR-151” as used herein refers to the compound I-2.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the disclosure exist as individual salts and hydrates, as well as a combination of, for example, a solvate of a salt of a compound of the disclosure.

As used in the present disclosure, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

In embodiments of the present disclosure, the compounds described herein may have at least one asymmetric center. Where compounds possess more than one asymmetric center, they may exist as diastereomers. It is to be understood that all such isomers and mixtures thereof in any proportion are encompassed within the scope of the present disclosure. It is to be further understood that while the stereochemistry of the compounds may be as shown in any given compound listed herein, such compounds may also contain certain amounts (for example, less than 20%, suitably less than 10%, more suitably less than 5%) of compounds of the present disclosure having an alternate stereochemistry. It is intended that any optical isomers, as separated, pure or partially purified optical isomers or racemic mixtures thereof are included within the scope of the present disclosure.

The compounds of the present disclosure may also exist in different tautomeric forms and it is intended that any tautomeric forms which the compounds form, as well as mixtures thereof, are included within the scope of the present disclosure.

The compounds of the present disclosure may further exist in varying polymorphic forms and it is contemplated that any polymorphs, or mixtures thereof, which form are included within the scope of the present disclosure.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “heterocycle” as used herein refers to a substituted or unsubstituted 5- or 6-membered heterocycle which can be aromatic or non-aromatic comprising at least one NH moiety.

The term “substituted” as used herein refers to when one or more available hydrogen on a compound is replaced with a non-hydrogen functional group.

The term “available”, as in “available hydrogen atoms” or “available atoms” refers to atoms that would be known to a person skilled in the art to be capable of replacement by a substituent.

The term “amine” or “amino,” as used herein, whether it is used alone or as part of another group, refers to groups of the general formula NR′R″, wherein R′ and R″ are each independently selected from hydrogen or C₁₋₆alkyl.

The term “subject” as used herein includes all members of the animal kingdom including mammals, and suitably refers to humans. Thus the methods and uses of the present disclosure are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt of a compound, wherein molecules of a suitable solvent are incorporated in the crystal lattice.

Prodrugs of the compounds of the present disclosure may be, for example, conventional esters formed with available hydroxy, thiol, amino or carboxyl groups. Other methods of forming prodrugs in general are known to a person skilled in the art and can be applied to the compounds of the present disclosure.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the disclosure to prevent recurrence. Treatment methods comprise administering to a subject a therapeutically effective amount of one or more of the compounds of the disclosure and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

The term “disease, disorder or condition” as used herein refers to a disease, disorder or condition mediated or treatable by activating SHIP1 such as by a compound of the disclosure.

The term “SHIP1” as used herein refers to Src homology 2-containing inositol 5′-phosphatase 1.

The term “mediated or treatable by activation of SHIP1” as used herein means that the disease, disorder or condition to be treated is affected by, modulated by and/or has some biological basis, either direct or indirect, that includes the presence in a cell of SHIP1 phosphatase. Such biological basis includes, for example, cytokines that are direct or indirect products of SHIP1 phosphatase. In an exemplary context, “activation of SHIP1” refers to an effect mediated through activation of the signaling in a cell or in an organism by SHIP1 for example via PIP₃ and/or IL-10.

As used herein, the term “effective amount” or “therapeutically effective amount” means an amount of one or more compounds of the disclosure that is effective, at dosages and for periods of time necessary to achieve the desired result. For example in the context of treating a disease, disorder or condition mediated or treatable by activation of SHIP1, an effective amount is an amount that, for example, increases the activity of SHIP1 compared to the activity of SHIP1 without administration of the one or more compounds.

The term “administered” as used herein means administration of a therapeutically effective amount of one or more compounds or compositions of the disclosure to a cell, tissue, organ or subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder.

The term “cancer” as used herein refers to cellular-proliferative disease states.

### II. Compounds and Compositions of the Disclosure

The present disclosure includes a compound for Formula I

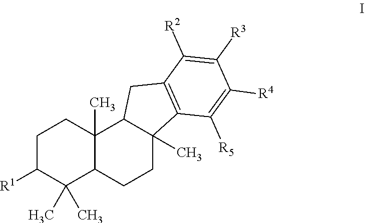

or a pharmaceutically acceptable salt, solvate, prodrug and/or derivative thereof,
wherein R¹ is selected from H, OH, OC(O)C₁₋₃alkyl, OC₁₋₃alkyl, NH₂, NHC₁₋₃alkyl, NHSO₂C₁₋₃alkyl, NSuccinamide, and NHC(O)C₁₋₃alkyl;
wherein R², R³, R⁴, and R⁵ are independently selected from H, OH, C₁₋₃alkyl, OC₁₋₃alkyl, NH₂, NHC₁₋₃alkyl, NHSO₂C₁₋₃alkyl, and NHC(O)C₁₋₃alkyl; or R² and R³, R³ and R⁴ or R⁴ and R⁵ taken together with the atoms they are attached to form a substituted or unsubstituted 5- or 6-membered heterocycle comprising at least one NH and optionally one or more additional heteroatoms selected from N, O, and S; and
wherein when R² and R³, R³ and R⁴ or R⁴ and R⁵ are taken together to form the substituted or unsubstituted 5- or 6-membered heterocycle, R⁴ and R⁵, R² and R⁵, or R² and R³ respectively are independently selected from H and C₁₋₃alkyl.

In some embodiments, the compound of Formula I is a compound of Formula IA

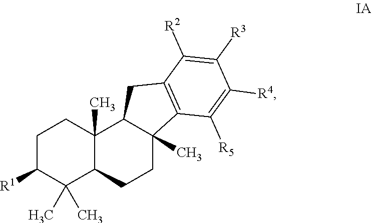

an enantiomer thereof, or a pharmaceutically acceptable salt, solvate, prodrug and/or derivative thereof.

In some embodiments, R¹ is selected from H, NH₂, NHC₁₋₃alkyl, NHSO₂C₁₋₃alkyl, NSuccinamide, and NHC(O)C₁₋₃alkyl.

In some embodiments, R² and R⁴ are H, and R³ and R⁵ are selected from OH, C₁₋₃alkyl, OC₁₋₃alkyl, NH₂, NHC₁₋₃alkyl, NHSO₂C₁₋₃alkyl, and NHC(O)C₁₋₃alkyl.

In some embodiments, R³ and R⁵ are selected from OH, CH₃, OCH₃, NHSO₂CH₃, and NHC(O)CH₃.

In some embodiments, R³ is selected from OH, OCH₃, NHSO₂CH₃, and NHC(O)CH₃; and R₅ is CH₃.

In some embodiments, R², R⁴, and R⁵ are H, and R³ is selected from OH, OC₁₋₃alkyl, NH₂, NHC₁₋₃alkyl, NHSO₂C₁₋₃alkyl, and NHC(O)C₁₋₃alkyl. For example, R³ is selected from OH, OCH₃, NHSO₂CH₃, and NHC(O)CH₃.

In some embodiments, the substituted or unsubstituted 5- or 6-membered heterocycle is aromatic or non-aromatic. In another embodiment, the 5- or 6-membered heterocycle comprises an NH moiety, and one more heteroatom selected from 0 and N. In a further embodiment, the substituents on the 5- or 6-membered heterocycle are selected from C═O and C₁₋₃alkyl.

In some embodiments, the substituted or unsubstituted 5- or 6-membered heterocycle are selected from

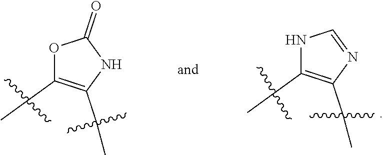 [SMILES: *c1[nH]c(=O)oc1* ; *c1nc[nH]c1*]

In some embodiments, R² and R³ taken together with the atoms they are attached to form the substituted or unsubstituted 5- or 6-membered heterocycle, and R⁴ and R⁵ are independently selected from H and C₁₋₃alkyl.

In some embodiments, the compound of Formula I is selected from

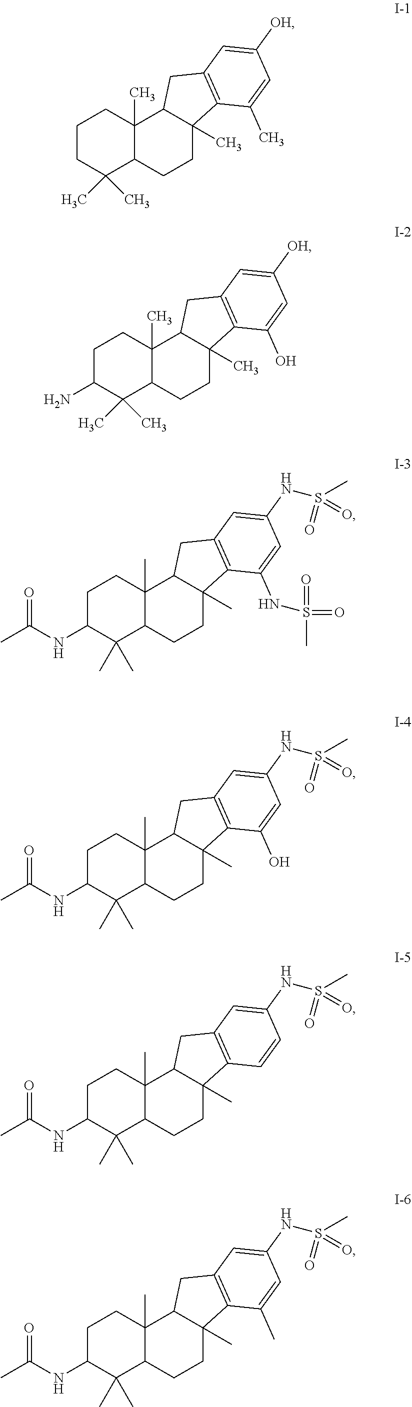

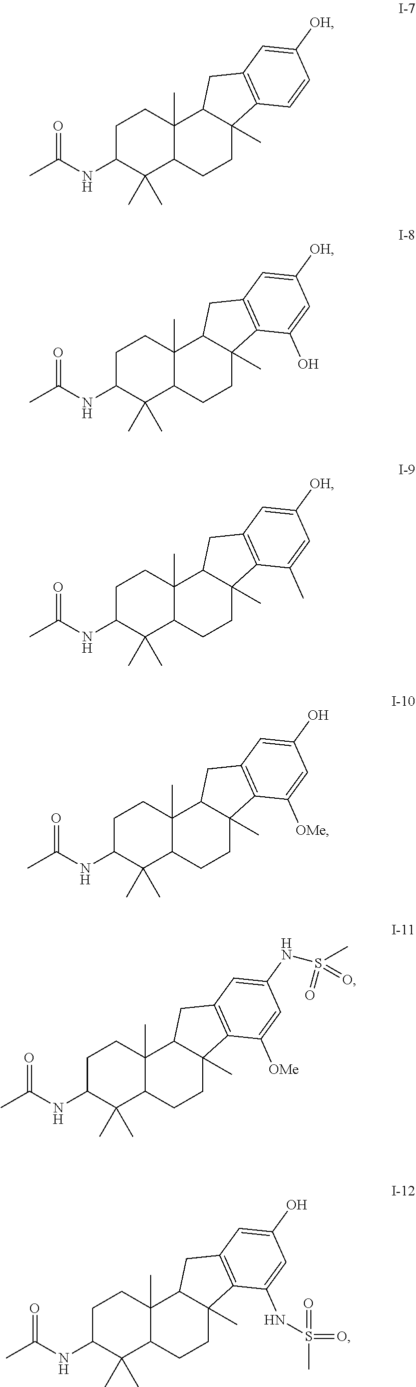

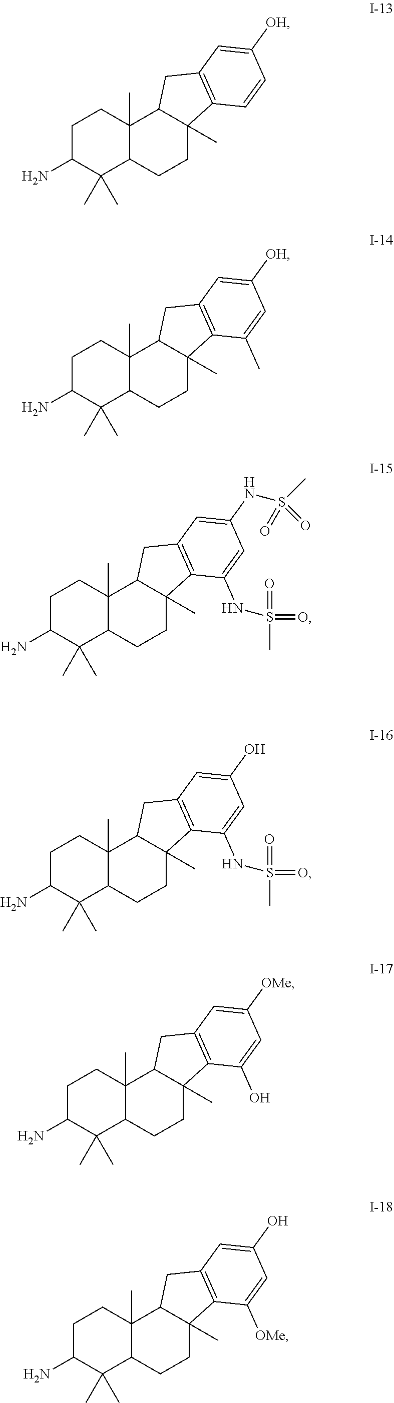 [SMILES: CC12CCC3C(C)(C)C(N)CCC3(C)C1Cc1cc(O)ccc12 ; Cc1cc(O)cc2c1C1(C)CCC3C(C)(C)C(N)CCC3(C)C1C2 ; CC12CCC3C(C)(C)C(N)CCC3(C)C1Cc1cc(NS(C)(=O)=O)cc(NS(C)(=O)=O)c12 ; CC12CCC3C(C)(C)C(N)CCC3(C)C1Cc1cc(O)cc(NS(C)(=O)=O)c12 ; COc1cc(O)c2c(c1)CC1C2(C)CCC2C(C)(C)C(N)CCC12C ; COc1cc(O)cc2c1C1(C)CCC3C(C)(C)C(N)CCC3(C)C1C2]

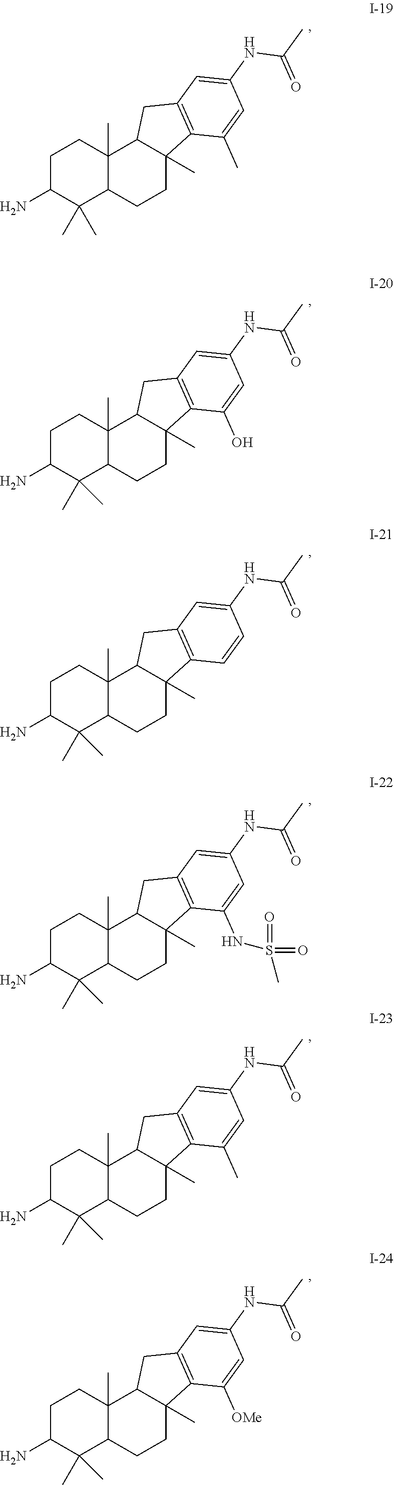 [SMILES: CC(=O)Nc1cc(C)c2c(c1)CC1C2(C)CCC2C(C)(C)C(N)CCC12C ; CC(=O)Nc1cc(O)c2c(c1)CC1C2(C)CCC2C(C)(C)C(N)CCC12C ; CC(=O)Nc1ccc2c(c1)CC1C2(C)CCC2C(C)(C)C(N)CCC12C ; CC(=O)Nc1cc2c(c(NS(C)(=O)=O)c1)C1(C)CCC3C(C)(C)C(N)CCC3(C)C1C2 ; CC(=O)Nc1cc(C)c2c(c1)CC1C2(C)CCC2C(C)(C)C(N)CCC12C ; COc1cc(NC(C)=O)cc2c1C1(C)CCC3C(C)(C)C(N)CCC3(C)C1C2]

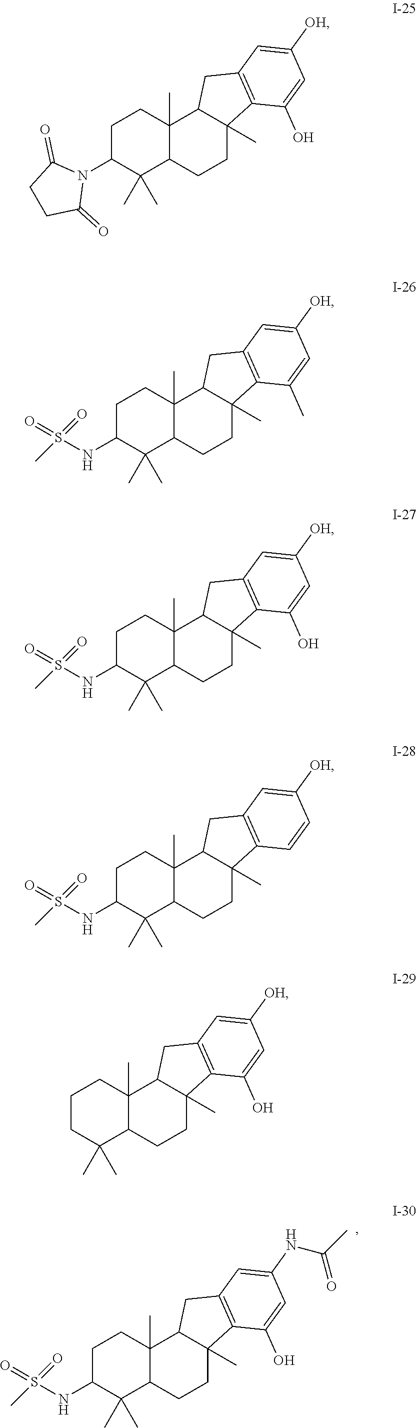 [SMILES: CC12CCC3C(C)(C)C(N4C(=O)CCC4=O)CCC3(C)C1Cc1cc(O)cc(O)c12 ; Cc1cc(O)cc2c1C1(C)CCC3C(C)(C)C(NS(C)(=O)=O)CCC3(C)C1C2 ; CC12CCC3C(C)(C)C(NS(C)(=O)=O)CCC3(C)C1Cc1cc(O)cc(O)c12 ; CC12CCC3C(C)(C)C(NS(C)(=O)=O)CCC3(C)C1Cc1cc(O)ccc12 ; CC1(C)CCCC2(C)C1CCC1(C)c3c(O)cc(O)cc3CC12 ; CC(=O)Nc1cc(O)c2c(c1)CC1C2(C)CCC2C(C)(C)C(NS(C)(=O)=O)CCC12C]

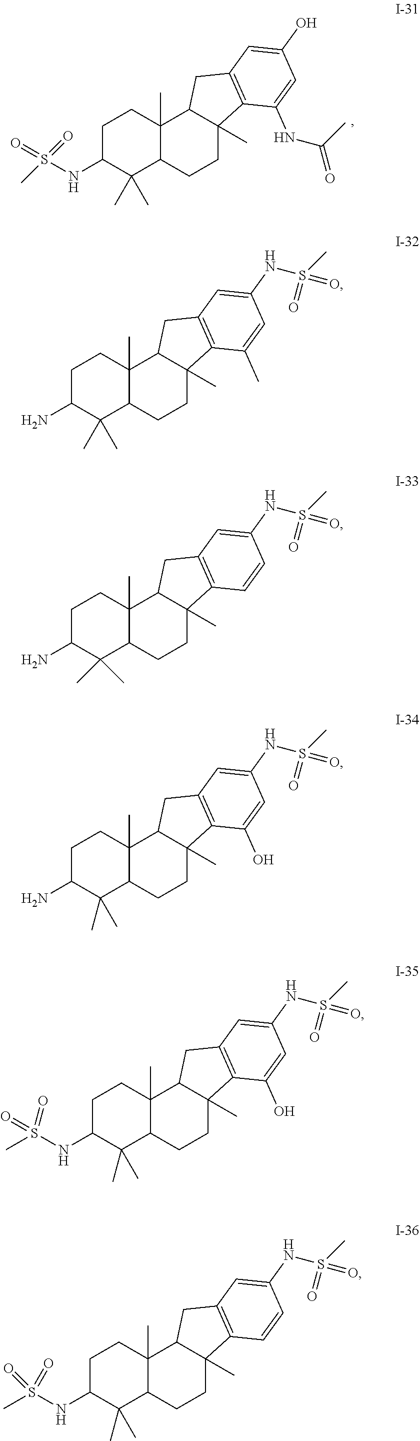

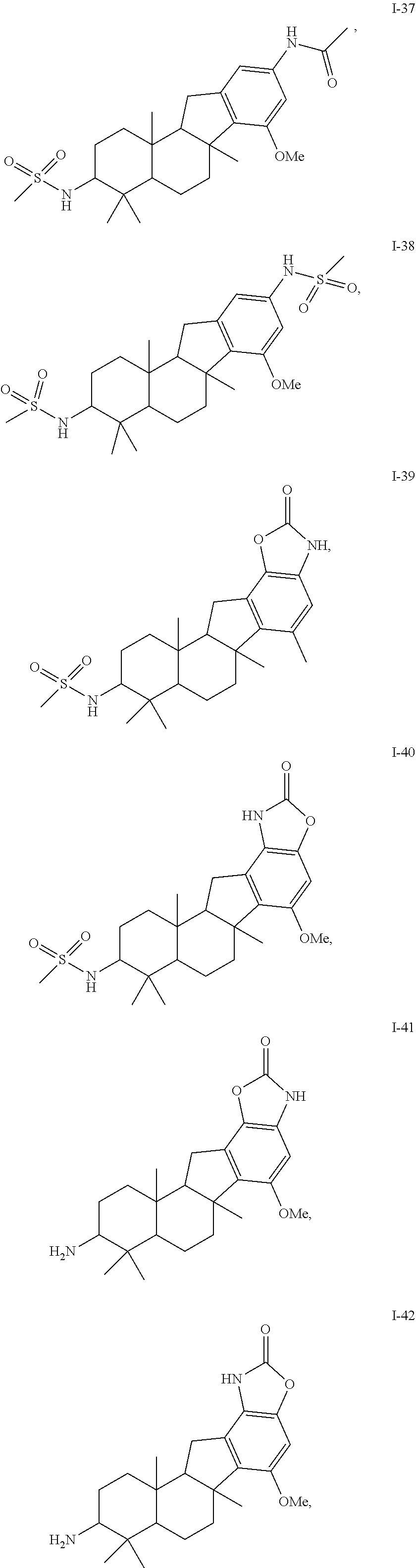 [SMILES: COc1cc(NC(C)=O)cc2c1C1(C)CCC3C(C)(C)C(NS(C)(=O)=O)CCC3(C)C1C2 ; COc1cc(NS(C)(=O)=O)cc2c1C1(C)CCC3C(C)(C)C(NS(C)(=O)=O)CCC3(C)C1C2 ; Cc1cc2[nH]c(=O)oc2c2c1C1(C)CCC3C(C)(C)C(NS(C)(=O)=O)CCC3(C)C1C2 ; COc1cc2oc(=O)[nH]c2c2c1C1(C)CCC3C(C)(C)C(NS(C)(=O)=O)CCC3(C)C1C2 ; COc1cc2[nH]c(=O)oc2c2c1C1(C)CCC3C(C)(C)C(N)CCC3(C)C1C2 ; COc1cc2oc(=O)[nH]c2c2c1C1(C)CCC3C(C)(C)C(N)CCC3(C)C1C2]

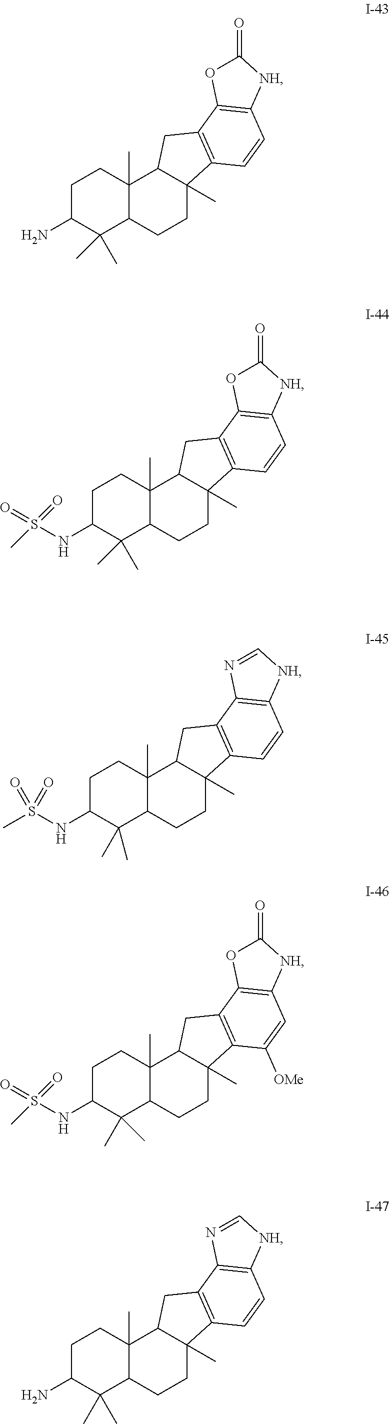 [SMILES: CC12CCC3C(C)(C)C(N)CCC3(C)C1Cc1c2ccc2[nH]c(=O)oc12 ; CC12CCC3C(C)(C)C(NS(C)(=O)=O)CCC3(C)C1Cc1c2ccc2[nH]c(=O)oc12 ; CC12CCC3C(C)(C)C(NS(C)(=O)=O)CCC3(C)C1Cc1c2ccc2[nH]cnc12 ; COc1cc2[nH]c(=O)oc2c2c1C1(C)CCC3C(C)(C)C(NS(C)(=O)=O)CCC3(C)C1C2 ; CC12CCC3C(C)(C)C(N)CCC3(C)C1Cc1c2ccc2[nH]cnc12]

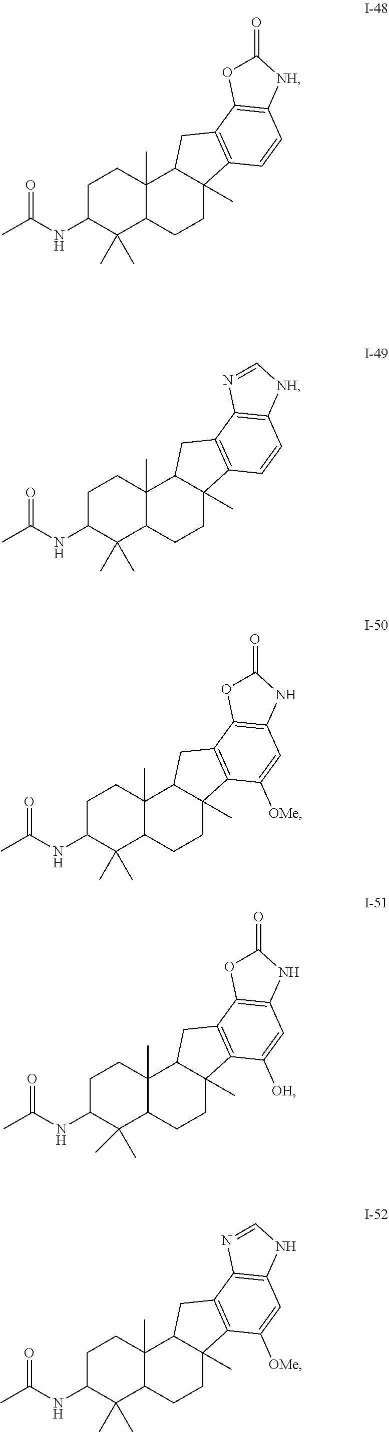 [SMILES: CC(=O)NC1CCC2(C)C3Cc4c(ccc5[nH]c(=O)oc45)C3(C)CCC2C1(C)C ; CC(=O)NC1CCC2(C)C3Cc4c(ccc5[nH]cnc45)C3(C)CCC2C1(C)C ; COc1cc2[nH]c(=O)oc2c2c1C1(C)CCC3C(C)(C)C(NC(C)=O)CCC3(C)C1C2 ; CC(=O)NC1CCC2(C)C3Cc4c(c(O)cc5[nH]c(=O)oc45)C3(C)CCC2C1(C)C ; COc1cc2[nH]cnc2c2c1C1(C)CCC3C(C)(C)C(NC(C)=O)CCC3(C)C1C2]

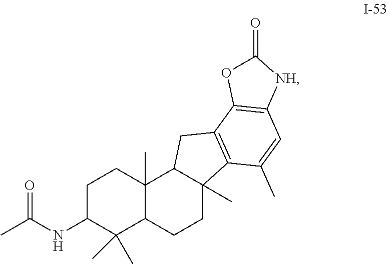 [SMILES: CC(=O)NC1CCC2(C)C3Cc4c(c(C)cc5[nH]c(=O)oc45)C3(C)CCC2C1(C)C]

and a pharmaceutically acceptable salt, solvate, prodrug and/or derivative thereof.

In some embodiments, the compound of Formula I of the present application does not include compound I-1. In some embodiments, the compound of Formula I of the present application does not include compound I-2. Optionally both compounds I-1 and I-2 are not included in the compound of Formula I.

In another aspect, the present disclosure includes a compound of the present disclosure or a pharmaceutically acceptable salt, solvate, prodrug and/or derivative for use in the treatment of a disease, disorder or condition mediated or treatable by activation of SHIP1.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the disclosure for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the disclosure include, for example, those made with solvents that are pharmaceutically acceptable. Examples of such solvents include water (resulting solvate is called a hydrate) and ethanol and the like. Suitable solvents are physiologically tolerable at the dosage administered.

The compounds of the present disclosure are suitably formulated in a conventional manner into compositions using one or more carriers. Accordingly, the present disclosure also includes a composition comprising one or more compounds of the disclosure and a carrier. The compounds of the disclosure are suitably formulated into pharmaceutical compositions for administration to subjects in a biologically compatible form suitable for administration in vivo. Accordingly, the present disclosure further includes a pharmaceutical composition comprising one or more compounds of the disclosure and a pharmaceutically acceptable carrier. In embodiments of the disclosure the pharmaceutical compositions are used in the treatment of any of the diseases, disorders or conditions described herein.

In some embodiments, the composition of the present disclosure consists essentially of one or more compounds of the present disclosure, or one or more pharmaceutically acceptable salts, solvates, prodrug and/or derivatives thereof, and a pharmaceutically acceptable carrier.

In some embodiments, the composition of the present disclosure consists of one or more compounds of the present disclosure, or one or more pharmaceutically acceptable salts, solvates, prodrug and/or derivatives thereof, and a pharmaceutically acceptable carrier.

The compounds of the disclosure are administered to a subject in a variety of forms depending on the selected route of administration, as will be understood by those skilled in the art. For example, a compound of the disclosure is administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the disclosure is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the disclosure is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the disclosure and use the lyophilizates obtained, for example, for the preparation of products for injection.

In some embodiments, a compound of the disclosure is administered parenterally. For example, solutions of a compound of the disclosure are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the disclosure are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a compound of the disclosure is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the disclosure are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the disclosure are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the disclosure and a suitable powder base such as lactose or starch. The aerosol dosage forms can also take the form of a pump-atomizer.

Compositions suitable for buccal or sublingual administration include tablets, lozenges, and pastilles, wherein a compound of the disclosure is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the disclosure are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, Pa., 1980, pp. 1530-1533 for further discussion of suppository dosage forms.

In some embodiments a compound of the disclosure is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the disclosure is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

A compound of the disclosure including pharmaceutically acceptable salts and/or solvates thereof is suitably used on their own but will generally be administered in the form of a pharmaceutical composition in which the one or more compounds of the disclosure (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### III. Methods and Uses of the Disclosure

In another aspect, the present disclosure includes a method of treating a disease, disorder or condition mediated or treatable by activation of SHIP1 comprising administering a therapeutically effective amount of a compound of the present disclosure or a pharmaceutically acceptable salt, solvate, prodrug and/or derivative thereof in a subject in need thereof.

In another aspect, the present disclosure includes a use of one or more compounds of the present disclosure or a pharmaceutically acceptable salt, solvate, prodrug and/or derivative thereof in the treatment of a disease, disorder or condition mediated or treatable by activation of SHIP1.

In another aspect, the present disclosure includes a use of one or more compounds of the present disclosure or a pharmaceutically acceptable salt, solvate, prodrug and/or derivative thereof in the manufacture of a medicament for the treatment of a disease, disorder or condition mediated or treatable by activation of SHIP1.

In some embodiments, the disease, disorder or condition mediated or treatable by activation of SHIP1 is selected from inflammatory bowel disease (IBD), multiple myeloma, allergy, a neoplastic disorder such as colon cancer, sepsis, organ injury, trauma, cardiovascular diseases, osteoporosis and sleep disorders. In some embodiments, the IBD is selected from Crohn's disease, and ulcerative colitis.

In some embodiments, the organ injury and trauma is mediated by IL-10 through SHIP1. In some embodiments, the organ injury and trauma is liver injury. In some embodiments, the liver injury is selected from viral hepatitis, autoimmune hepatitis, primary biliary cirrhosis, and liver allograft rejection. It is known that IL-10 administration reduces organ injury such as liver or lung inflammation, and reduces neuropathy in neural or spinal cord injury.

In some embodiments, the cardiovascular diseases include atherosclerosis. It is known that IL-10 administration limits tissues inflammation and improves endothelial and macrophage function.

In some embodiments, the osteoporosis disorders include those that IL-10 administration can inhibit resorptive function of mature osteoclast.

In some embodiments, the disease, disorder or condition mediated or treatable by activation of SHIP1 is multiple myeloma.

In some embodiments, the sepsis is severe sepsis. It is known that viral infections such as COVID-19 causes severe sepsis. In some embodiments, the severe sepsis is caused by COVID-19.

In an embodiment, the treatment is in an amount effective to ameliorate at least one symptom of the neoplastic disorder, for example, reduced cell proliferation or reduced tumor mass, among others, in a subject in need of such treatment.

Neoplasms can be benign (such as uterine fibroids and melanocytic nevi), potentially malignant (such as carcinoma in situ) or malignant (i.e. cancer). Exemplary neoplastic disorders include the so-called solid tumours and liquid tumours, including but not limited to carcinoma, sarcoma, metastatic disorders (e.g., tumors arising from the prostate), hematopoietic neoplastic disorders, (e.g., leukemias, lymphomas, myeloma and other malignant plasma cell disorders), metastatic tumors and other cancers.

The disclosure further includes one or more compounds of the disclosure for use in treating cancer. In an embodiment, the compound is administered for the prevention of cancer in a subject such as a mammal having a predisposition for cancer.

In an embodiment, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In a further embodiment, the disease, disorder or condition mediated or treatable by activation of SHIP1 and the one or more compounds of the disclosure are administered in combination with one or more additional cancer treatments. In another embodiment, the additional cancer treatment is selected from radiotherapy, chemotherapy, targeted therapies such as antibody therapies and small molecule therapies such as tyrosine-kinase and serine-threonine kinase inhibitors, immunotherapy, hormonal therapy and anti-angiogenic therapies.

In an embodiment, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

In an embodiment, the compounds of the disclosure are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the disclosure, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In an embodiment, the subject is a mammal. In another embodiment, the subject is human.

Compounds of the disclosure are either used alone or in combination with other known agents useful for treating diseases, disorders or conditions that are mediated or treatable by activation of SHIP1, and those that are treatable with a SHIP1 agonist, such as the compounds disclosed herein. When used in combination with other agents useful in treating diseases, disorders or conditions mediated or treatable by activation of SHIP1, it is an embodiment that a compound of the disclosure is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present disclosure that a combination of agents is administered to a subject in a non-contemporaneous fashion. In an embodiment, a compound of the present disclosure is administered with another therapeutic agent simultaneously or sequentially in separate unit dosage forms or together in a single unit dosage form. Accordingly, the present disclosure provides a single unit dosage form comprising one or more compounds of the disclosure, an additional therapeutic agent, and a pharmaceutically acceptable carrier.

The dosage of a compound of the disclosure varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, a compound of the disclosure is administered initially in a suitable dosage that is adjusted as required, depending on the clinical response. Dosages will generally be selected to maintain a serum level of the compound of the disclosure from about 0.01 μg/cc to about 1000 μg/cc, or about 0.1 μg/cc to about 100 μg/cc. As a representative example, oral dosages of one or more compounds of the disclosure will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg.

### EXAMPLES

The following non-limiting examples are illustrative of the present disclosure.

### General Methods

Mouse colonies. BALB/c mice wild type (_{+/+}) or SHIP1 knockout (_{-/-}) mice were provided by Dr. Gerald Krystal (BC Cancer Research Centre, Vancouver, BC). The generation of STAT3_{-/-}mice started with crossing C57BL/6 STAT3 flox/flox mice (Dr. Shizuo Akira, Hyogo College of Medicine, Nishinomiya, Japan) with C57BL/6 LysMcre mice (Jackson Laboratory). Their offspring were then crossed with homozygous STAT3 flox/flox mice to produce to generate both STAT3 flox/flox/LysMCre_{+/-}mice (referred to be STAT3_{-/-} mice) and STAT3 flox/flox mice (STAT3_{+/+}mice) in the same litters. All mice were maintained in accordance with the ethic protocols approved by the University of British Columbia Animal Care Committee.

Constructs. The mammalian (lentiviral) expression plasmids of SHIP1 in FUGWBW were generated using Gateway LR reactions from pENTR1A (Invitrogen, Burlington, ON) constructs. A pENTR1A-His6-SHIP1 WT (SHIP1 Uniprot ID Q9ES52) plasmid was used as the template for standard primer based, site-directed mutagenesis to generate the K681A, Y190F, Y799F, Y659F and Y657F mutants. The phosphatase disrupted SHIP1 construct (P671A, D675A, and R676G in the phosphatase domain) was kindly provided by Dr. KS Ravichandran (University of Virginia). The constructs were confirmed by DNA sequencing. Subsequently, a Gateway LR reaction was performed between pENTR1A construct and FUGWBW (FUGW in which the green fluorescent protein was replaced by the Gateway cassette, and a blasticidin S resistance gene expression cassette was inserted downstream of the Gateway cassette (Peacock et al., 2009). Success of the LR reaction was confirmed by restriction enzyme digest. For Clover/mRuby2 based FRET experiments (Lam et al., 2012), pENTR1A Clover-SHIP1 was constructed by inserting a Clover fragment from pcDNA3 Clover (Addgene) to the N-terminal of SHIP1 in pENTR1A-His6-SHIP1 WT, replacing the His6.pENTR1A STAT3-mRuby2 was constructed by cloning murine STAT3 (Uniprot ID P42227) into pENTR1A followed by insertion of a mRuby2 fragment from pcDNA3 mRuby2 (Addgene) to the N-terminus of STAT3. Constructs were confirmed by sequencing and transferred to FUGWBW as above.

Bacteria expression vectors to produce recombinant proteins for crystallography and biolayer interferometry were generated by ligase-independent cloning (LIC) methodology in the LIC-HMT vector (Van Petegem et al., 2004). This plasmid contains an N-terminal tag composed of His6 and maltose binding protein (MBP), followed by a TEV protease cleavage site (abbreviated as the HMT-tag). The PCR product was purified and treated with T4 DNA polymerase (LIC-quality) (Novagen, Madison, Wis.) in the presence of dCTP only. The LIC-HMT vector was digested with SspI and the linearized plasmid was treated with T4 DNA polymerase in the presence of dGTP only. Equal volumes of insert and vector were mixed and incubated at room temperature for 10 minutes, followed by transformation into chemical competent E. coli DH5a cells using the standard heat shock protocol, and selection on kanamycin-containing LB agar plates. To generate different PAC2 mutants, standard site-directed mutagenesis was employed. Identities of all plasmids were confirmed by DNA sequencing.

Cell lines. J16 and J17 cell lines derived from SHIP1_{+/+}and _{-/-}BMDM respectively were previously described (Ming-Lum et al., 2012) and cultured in Mac media (IMDM supplemented with 10% (v/v) FCS, 10 μM β-mercaptoethanol, 150 μM monothioglycolate and 1 mM L-glutamine). J17 cells expressing wild type and mutant His₆-SHIP1, Clover-SHIP1 or mRuby2-STAT3 constructs were generated by lentivirus mediated gene transfer as described (Cheung et al., 2013). Transduced cells were selected with 5 μg/ml blasticidin. Clover-SHIP1 and mRuby2-STAT3 cells were further subjected to fluorescent activated cell sorting to select the brightest cells on a FACS Aria II cytometer.

Isolation of mouse peritoneal macrophages. Primary peritoneal macrophages (perimacs) were isolated from mice by peritoneal lavage with 3 ml of sterile Phosphate Buffered Saline (PBS) (Thermo Fisher Scientific, Nepean, ON). Perimacs were collected and transferred to Mac media.

Production of bone marrow-derived macrophages. Bone marrow-derived macrophages (BMDMs) were generated by first collecting femurs and tibias from mice, and then flushing out the bone marrow through a 26-G needle. Extracted cells were plated, in Mac media supplemented with 5 ng/ml each of CSF-1 and GM-CSF (Stem Cell Technologies, Vancouver, BC), on a 10-cm tissue culture plate for 2 hours at 37° C. Non-adherent cells were collected and replated at 9×10⁶ cells per 10-cm tissue culture plate. Cells were then cultured in the presence of CSF-1 and GM-CSF. Differentiated BMDMs were used after 7 to 8 days. All cells were maintained in a 37° C., 5% CO2, 95% humidity incubator.

Continuous Flow Cultures. The continuous flow apparatus facilitates constant stimulation and removal of cell supernatants to determine kinetic profiles of cytokine production over time. BMDMs were seeded at 3×10₆ cells per well in a 24-well tissue culture plate that had been coated with poly-L-lysine (Thermo Fisher Scientific, Nepean, ON) and rinsed with PBS. After overnight incubation, culture media was removed and Leibovitz's L-15 (L-15) media (Invitrogen, Burlington, ON) supplemented with 3% FCS, 10 μM β-mercaptoethanol and 150 μM monothioglycolate was added. Cells were allowed to equilibrate in L-15 media for 1 hour before being placed in the continuous flow apparatus. Stimulation solution was made in the same media equilibrated at 37° C., and was passed through a modified inlet fitted to the well by a syringe pump (New Era Syringe Pumps Inc., Farmingdale, N.Y.). A flow rate of 150 μl per minute was used. At the same time, cell supernatants were removed from the well at the same flow rate, and fractions were collected at 5-minute intervals over the course of 3 hours. These fractions were analyzed for secreted TNFα levels by ELISA.

Real-time quantitative PCR. Total RNA was extracted using Trizol reagent (Invitrogen, Burlington, ON) according to manufacturer's instructions. About 2-5 μg of RNA were treated with DNAsel (Roche Diagnostics, Laval, QC) according to the product manual. For mRNA expression analysis, 120 ng of RNA were used in the Transcriptor First Strand cDNA synthesis kit (Roche Diagnostics, Laval, QC), and 0.1 μl to 0.2 μl of cDNA generated were analyzed by SYBR Green-based real time PCR (real time-PCR) (Roche Diagnostics, Laval, QC) using 300 nM of gene-specific primers. Expression levels of mRNA were measured with the StepOne Plus RT-PCR system (Applied Biosystems, Burlington, ON), and the comparative Ct method was used to quantify mRNA levels using GAPDH as the normalization control.

Measurement of TNFα production. Cells were seeded at 50×10⁴ cells per well in a 96-well tissue culture plate and allowed to adhere overnight. Media was changed the next day 1 hour prior to stimulation. Cells were stimulated with 1 or 10 ng/ml LPS+/−various concentrations of IL10 for 1 hour. Supernatant was collected and secreted TNFα protein levels were measured using a BD OptEIA Mouse TNFα Enzyme-Linked Immunosorbent Assay (ELISA) kit (BD Biosciences, Mississauga, ON). Triplicates wells were used for each stimulation condition. IC50 values were calculated from three independent experiments and differences in IL10 1050 values from cells expressing SHIP1 mutants vs SHIP1 WT protein were analyzed by one-way ANOVA.

In vitro phosphatase assay. The phosphatase assay was performed in 96-well microtiter plates with 10 ng of enzyme/well in a total volume of 25 μL in 20 mM Tris-HCl, pH 7.4, 150 mM NaCl, 0.05% Tween-20, 10 mM MgCl₂ as described (Ong et al., 2007). Enzyme was incubated with or compound I-1 (dissolved in ethanol) for 10 minutes at 23° C., before the addition of 50 μM of inositol-1,3,4,5-tetrakisphosphate (IP4) (Echelon Bioscience Inc., Salt Lake City, Utah). The reaction was allowed to proceed for 10 minutes at 37° C. and the amount of inorganic phosphate released was assessed by the addition of Malachite

Green reagent and absorbance measurement at 650 nm. For enzyme kinetic determination, different concentrations of IP₄ (0-300 μM) were used and the reactions were stopped at different time points. Initial velocities were calculated, and Kcat and KM were determined using GraphPad 6 software.

In vitro pull down assay. J17 His6-SHIP1 and Y190F cells were seeded at 2×10⁶ cells per well in a 6-well plate. After overnight incubation, fresh cell media was added for 30 minutes before stimulation with 100 ng/ml IL10, 1 L6 or 20 μM compound I-2 for 5 minutes. Cells were lysed with Protein Solubilization Buffer (PSB, 50 mM Hepes, pH 7.5, 100 nM NaF, 10 mM Na Pyrophosphate, 2 mM NaVO4, 2 mM Na Molybdate, 2 mM EDTA) containing 1% octylglucoside, 0.01 M imidazole, and protease inhibitor cocktail (Roche Diagnostics, Laval, QC) for 30 minutes at 4° C. and centrifuged at 10000 rpm for 15 minutes. EDTA resistant Ni beads (Roche Diagnostics, Laval, QC) were added to supernatants and the mixture incubated at 4° C. for 1 hour before spinning down and washing of beads three times with 0.1% octylglucoside wash buffer in PSB. Bead samples and starting material cell lysates were separated on a 7.5% SDS-PAGE gel.

Immunoblotting. Protein lysates were separated on SDS-PAGE gels and transferred onto polyvinylidene fluoride (PVDF) membrane (Millipore, Etobicoke, ON). Membranes were blocked and probed, where appropriate, with the following primary antibodies overnight: SHIP1 (P1C1) (Santa Cruz Biotechnology), pSHIP1 (Y190) (Kinexus), STAT3 (9D8) (ThermoFisher Scientific), pSTAT3 (Y705) (Thermo Fisher Scientific), STAT1 (BD Transduction Laboratories), pSTAT1 (Y701) (Upstate Biotechnology), GAPDH and actin (Sigma-Aldrich). Membranes were developed with either Alexa Fluor® 660 anti-mouse IgG or Alexa Fluor® 680 anti-rabbit IgG antibodies (Thermo Fisher Scientific) and imaged using a LI-COR Odyssey Imager.

Acceptor Photobleaching FRET analysis. J17 cells expressing Clover-SHIP1 and/or mRuby2-STAT3 were seeded at 50×10₄ cells per well in 8-well Ibidi p-Slides (Ibidi GmbH, Martinsried, Germany). After overnight incubation, cells were serum-starved with Mac media containing 1% serum for 3 hours before media replacement with Leibovitz's (L-15) media (Invitrogen, Burlington, ON) supplemented with 1% serum, 10 μM β-mercaptoethanol, 150 μM monothioglycolate and 1 mM L-glutamine for confocal microscopy imaging. Cells were imaged on a Leica SP8X on DMi8 confocal microscope system with a 63×/1.3 Gly HC PL APO CS2 objective using a white light laser line with 488 nm for donor excitement and 555 nm for acceptor excitement. Photobleaching FRET analysis was performed by measuring Clover-SHIP1 donor fluorescence intensity before and after bleaching the acceptor, mRuby2-STAT3, within a field of cells ‘region of interest’ (ROI), at 100% laser intensity for 60 frames. Acceptor photobleaching was performed first in resting cells then at 1 minute (±5 seconds) following ‘mock’ stimulation with L-15 media, or L-15 media containing 100 ng/ml IL10, IL6 or 20 μM compound I-2. Donor and acceptor fluorescence intensity of individual cells within the bleached ROI was quantified before and after bleaching. Percentage FRET efficiency was calculated as % FRETeff=100×(Dₚₒₛₜ−Dₚᵣₑ)/Dₚₒₛₜ where Dₚᵣₑ and Dₚₒₛₜ represent Clover-SHIP1 donor fluorescence intensity before and after bleaching, respectively.

Immunofluorescence. Perimacs were seeded at 3×10⁵ cells per well in 18-well Ibidi p-Slides (Ibidi GmbH, Martinsried, Germany) and allowed to adhere for 3 hours before washing with PBS to remove non-adherent cells. CD8+ T cells were seeded at 2×10⁶ cells in 12-well tissue culture plates. Cells were stimulated with either 100 ng/ml IL10 or 20 μM compound I-2 for 2 or 20 minutes followed by 3×PBS washes and fixing of cells in 4% paraformaldehyde for 15 minutes at room temperature. Cells were incubated with anti-mouse CD16/CD32 Fc Block (BD Pharmingen) for 1 hour followed by an overnight incubation at 4° C. with either anti-SHIP1 antibody (P1C1) (Santa Cruz Biotechnology) or anti-STAT3 antibody (9D8) (ThermoFisher Scientific). Cells were then incubated with anti-mouse IgG (H+L)-Alexa Fluor 660 secondary antibody (ThermoFisher Scientific) for 1 hour, followed by, for perimacs, anti-CD11b-FITC antibody (BD Pharmingen) for 30 minutes. CD8+ T cells were mounted onto 18-well Ibidi p-Slides prior to confocal microscopy and cells were stored in Ibidi Mounting Media supplemented with ProLong Gold antifade reagent with DAPI (Molecular Probes, Life Technologies). Cells were imaged on a Leica SP5II on DM6000 confocal microscope with a 63×/1.4-0.6 Oil PL APO objective using 405, 488 and 633 nm laser lines for excitation. Final images were scanned sequentially acquiring eight Z-stacks with a frame-average of four. Co-localization analysis was performed using ImageJ software by first combining individual z-stack confocal images then performing deconvolution and co-localization using CUDA deconvolution and JACoP plugins respectively. Pearson's coefficient values were produced as a measurement of the degree of overlap between SHIP1 or STAT3 with CD11b (for Perimacs) or DAPI.

Mouse Endotoxemia Model. Groups of 6-8 week old BALB/c SHIP1_{+/+} and SHIP1_{-/-}mice were intraperitoneally injected with either 1 or 5 mg/kg of LPS with or without co-administration of 1 mg/kg of IL10. Blood was drawn 1 hour later by cardiac puncture for determination of plasma cytokine levels by ELISA. ELISA kits were purchased from BD Biosciences (Mississauga, ON) for TNFα.

Mouse Colitis Model. Colitis was induced in 6-8 week old BALB/cIL10_{-/-}mice by administering the colonic contents of conventional C57BL/6 mice diluted 1:10 in PBS by oral gavage. Mouse weights and fecal consistencies were monitored and colitis allowed to develop for 6 weeks. Ethanol (Vehicle) and compound I-1 (3 mg/kg) was diluted in cage drinking water and dexamethasone (0.4 mg/kg) was administered every 2 days by oral gavage for 3 weeks. At the end of the dosing period, proximal, medial and distal colon sections were collected for paraffin embedding or stored in RNALater (Invitrogen, Mississauga, ON) for RNA extraction. Slides were prepared, stained with hematoxylin and eosin, and mounted by the UBC Department of Pathology and Laboratory Medicine Histochemistry Facility. Specimens were assigned pathological scores by two, independent, blinded investigators according to a method described by Madsen et al (Madsen et al., 2001). In brief, colonic inflammation was graded using a 4-point scale, scoring 0-3 for each of submucosal edema, immune cell infiltration, goblet cell ablation, and integrity of the epithelial layer. For analysis of mRNA expression, colon sections were homogenized and total RNA extracted as described above and analyzed by real time PCR using gene specific primers for IL17, CCL2, and GAPDH (normalization control).

Expression of PAC2 for crystallography. LIC-HMT-PAC2 expression vector was transformed into E. coli Rosetta(DE3) pLacI cells. Overnight culture was inoculated with a 250-fold dilution to start the actual culture. The cells were grown at 37° C. in LB medium (supplemented with 50 μg/ml of kanamycin and 34 μg/ml of chloramphenicol) with shaking at 225 rpm. When OD₆₀₀ reached about 0.6, the culture was cooled down to room temperature before the addition of 0.4 mM isopropyl β-D-1-thiogalactopyranoside (IPTG) to induce the expression of recombinant protein. Cultures were left in the shaker overnight (usually 16-18 hours) at 22° C., and then collected by centrifugation (5000 g for 10 minutes at 4° C.). The cell pellet was subsequently resuspended in lysis buffer (20 mM Tris-HCl pH 7.4, 350 mM NaCl, 10 mM TCEP, 5 mM imidazole, supplemented with 1×EDTA-free Protease Inhibitor Cocktail (PIC) (Roche Diagnostics, Laval, QC) and 25 μg/ml lysozyme), and lysed via sonication (2 cycles of 2 minutes pulse) on ice. Cell debris was removed by two rounds of centrifugation, first at 5000 g for 15 minutes at 4° C. followed by 18000 rpm for 30 minutes at 4° C. Supernatant was filtered with a 0.45 μm filter and loaded onto a Talon Co₂₊-affinity column, previously equilibrated with Buffer A (20 mM Tris-HCl pH 7.4, 250 mM NaCl, 1 mM TCEP), and washed with 10 column volume (CV) of Buffer B (Buffer A+5 mM imidazole). Bound proteins were eluted with 6 CV of Buffer C (Buffer A+50 mM imidazole).

To remove the HMT tag, TEV protease (purified in house as a His6-tagged protein) was added to the eluted protein, which was then dialyzed against Buffer D (20 mM Tris-HCl pH 7.4, 250 mM NaCl, 1 mM TCEP) overnight at 4° C. with gentle stirring. The dialyzed sample was loaded onto the Amylose column (New England Biolabs, Whitby, ON), and the flow through, which contained the untagged protein, was loaded onto the Talon column to remove the His6-TEV protease. The flow through from the Talon column was dialyzed against Buffer E (20 mM Tris-HCl pH 7.4, 25 mM NaCl, 1 mM TCEP) overnight at 4° C. with gentle stirring, and then loaded onto the ResourceQ column (6 ml column volume) (GE Healthcare, Mississauga, ON), followed by washes with 3 CV of Buffer E. To elute the protein, Buffer F (20 mM Tris-HCl pH 7.4, 1000 mM NaCl, 1 mM TCEP) was used. A gradient from 25 mM NaCl (0% buffer G) to 200 mM NaCl (20% Buffer G) was used across 20 CV to separate the components in the protein sample. The fractions were analyzed by SDS-PAGE. PAC2 usually eluted from the ResourceQ column at ˜130 mM NaCl. The purified protein was concentrated to about 5-10 mg/ml using Amicon concentrators with 30K MWCO (Millipore, Etobicoke, ON), and exchanged into the desired buffer. For protein crystallization, the desired buffer contained 50 mM Tris-HCl pH7.4, 25 mM NaCl and 0.5 mM TCEP. For Biolayer Interferometry, HMT-PAC2 proteins eluted from the first Talon column were directly purified on the ResourceQ column without cleavage of the HMT tag.

Expression of PAC2-Avi tag for Biolayer interferometry. A sequence corresponding to Avi-tag (GLNDIFEAQKIEWHE) was added to the c-terminal end of the PAC2 in LIC-HMT-PAC2 expressing vector via standard restriction digestion and ligation. The LIC-HMT-PAC2-Avi expressing vector was then co-transformed into E. coli BL21 cells with pBirAcm expression vector in 1:1 molar ratio. Overnight culture was inoculated with a 250-fold dilution to start the actual culture. The cells were grown at 37° C. in LB medium (supplemented with 50 μg/mL of kanamycin and 10 μg/mL of chloramphenicol) with shaking speed of 225 rpm. When OD₆₀₀ reached about 0.6, 5 mM of biotin in bicine buffer pH 8.3 was added to the culture to have final concentration of 125 μM of biotin. The culture was then cooled down to room temperature before the addition of 0.4 mM isopropyl-B-D-1-thiogalactopyranoside (IPTG) to induce the expression of the recombinant protein with Avi-tag. The rest of the method is identical as written in “Expression of PAC2 for crystallography”.

Protein crystallization, data collection, phasing and refinement. Initial crystallization hits were obtained via sparse matrix screening in 96-well plates using commercially available crystallographic solutions (Qiagen, Toronto, ON). Optimization of crystallization conditions was performed in 24-well plate format using the hanging drop vapor diffusion method. Diffraction-quality protein crystals were obtained at 4-7 mg/ml protein at room temperature with 0.1 M HEPES-NaOH pH 6.7, 20% PEG1500 and 5 mM MgCl₂. The PAC2-cc protein contained surface entropy reduction mutations (E770A, E772A, E773A) and aided in improving crystal quality. Unique fragments of crystal clusters of protein were soaked for 5 to 10 seconds in the crystallization solution containing 25% isopropanol, and flash-frozen in liquid nitrogen.

Diffraction data set were collected at the Advance Proton Source (APS) beamline 23-ID-D-GM/CA and processed with XDS through XDSGUI₄₅. The phase problem was solved with an unpublished structure as search model in Phaser MR ₄₇. The initial model was refined with COOT ₄₈ and Refmac5 ₄₉. Towards the final model occupancy refinement of sidechains was used in Phenix(Adams et al., 2010) and three TLS groups were defined. Data collection and refinement statistics are shown in Table 1. The model and data were deposited under protein database ID 6DLG.

Small angle X-ray scattering. PAC1 samples in 190 μM in 50 mM TrisHCl (pH7.4) 150 mM NaCl, 1 mM TCEP and 2% EtOH with and without 570 μM compound I-1 (6 fold molar excess). Dynamic Light Scattering (DLS) data for PAC1 and PAC1-compound I-1 complexes were collected prior to SAXS data collection to confirm that all the samples are highly pure and suitable for data collection. The data collection was performed on a 3-pinhole camera (S-MAX3000; Rigaku Americas, The Woodlands, Tex.) equipped with a Rigaku MicroMaxp002 microfocus sealed tube (Cu Kα radiation at 1.54 Å) and a Confocal Max-Flux (CMF) optics system operating at 40 W (Rigaku). Scattering data were recorded using a 200 mm multiwire two-dimensional detector. The data for both samples and buffer were collected for 3 h for each sample within the range of 0.008≤s≤0.26 Å-1 and processed according to the method previously described, where s=4π sin θ/λ (Patel et al., 2011, Patel et al., 2010, Patel et al., 2012). The Normalized Spatial Discrepancy (NSD) of the non-liganded and liganded PAC1 models were 0.6 and 1.0 respectively.

Biolayer interferometry. The binding affinity between the PAC2 protein and small molecule allosteric regulators was examined via bio-layer interferometry (BLI) experiments using super-streptavidin (SSA) biosensor tips and an Octet Red 96 instrument (ForteBio, Fremont, Calif.). SSA biosensor tips were hydrated in assay buffer 20 mM Tris-HCl (pH 7.4), 150 mM NaCl, 10 mM MgCl₂, 0.5 mM TCEP, 0.2% Tween-20 prior to protein immobilization. 0.5 ug/mL of protein was immobilized to the SSA biosensor overnight at 4° C. After immobilization of protein to the biosensor, the tips were blocked with 0.1% BSA for 90 minutes followed by 20 minutes of wash with assay buffer supplemented with 1% EtOH. The kinetic measurement was done at 30° C. with orbital flow of 1,000 RPM. The baseline was achieved with the assay buffer supplemented with 1% EtOH for 60 s. The association was measured for 600 s at an analyte concentration of 20 μM followed by dissociation for 300 s in the same buffer as the baseline. The raw data was analyzed using the Octet Red Data Analysis software (ver. 8.2). The raw data were aligned to the baseline and subtracted using both single and double reference subtraction.

### Quantification and Statistical Analysis

The band quantification of all immunoblots were performed using LI-COR Odyssey imaging system and Image Studio_{TM}Lite software (LI-COR Biosciences, Lincoln, Nebr.). GraphPad Prism 6 (GraphPad Software Inc., La Jolla, Calif.) was used to perform all statistical analyses. Statistical details can be found in figure legends. Values are presented as means±standard deviations. Unpaired t tests were used where appropriate to generate two-tailed P values, One-way or Two-way ANOVA were performed where required with appropriate multiple comparisons tests. Differences were considered significant when p≤0.05.

### Data and Software Availability

The X-ray crystallography data that supports the findings of this study has been deposited in the Worldwide Protein Data Bank (wwPDB) under the ID code, PDB ID 6DLG.

### Formulation of Compounds

50 mg/mL Stocks of compound I-1 in 50% Cremophor EL/50% ethanol v/v was made according the following general procedure. Equal volumes of 100 mg/mL compound I-1 in Cremophore EL and ethanol were mixed together by pipetting (around 50 times or more to ensure only one phase is visible). Stock solutions of other compounds were made in a similar manner. The stock solutions were kept at 4° C. Stock solution can be warmed to room temperature before making working solution (see below)

5 mg/mL solutions of compound I-1 with 5% v/v Cremophore EL/5% v/v ethanol in PBS (phosphate buffer saline) were made according to the following general procedure. The volume of 50 mg/mL stock solution of compound I-1 in 50% Cremophor EL/50% ethanol v/v needed was calculated in addition to the volume of water. To the volume required of the 50 mg/mL stock solution, an equal amount of water was added and mixed well by pipetting. The pipet tip used for the stock solution is rinsed repeatedly with small portions of the remaining volume of water until the pipet tip was clean and the desired total volume of the 5 mg/mL solution was achieved. The resulting solution was mixed well until homogenous.

### Administration to Mice in Drinking Water

The mice used were 30 g each and drank around 2 mL of water per day.

The IBD model mice were dosed with 2 or 3 mg/kg of compounds of the present disclosure, for example compound I-1. The MM model mice were dosed with about 20 to about 50 mg/kg of compounds of the present disclosure, for example compound I-1. The appropriate amount of 5 mg/mL PBS solution with 5% v/v Cremophore EL/5% v/v ethanol as prepared above was diluted into the mice's drinking water.

### Example 1 IL10 Requires Both SHIP1 and STAT3 to Inhibit Macrophage Production of TNFα

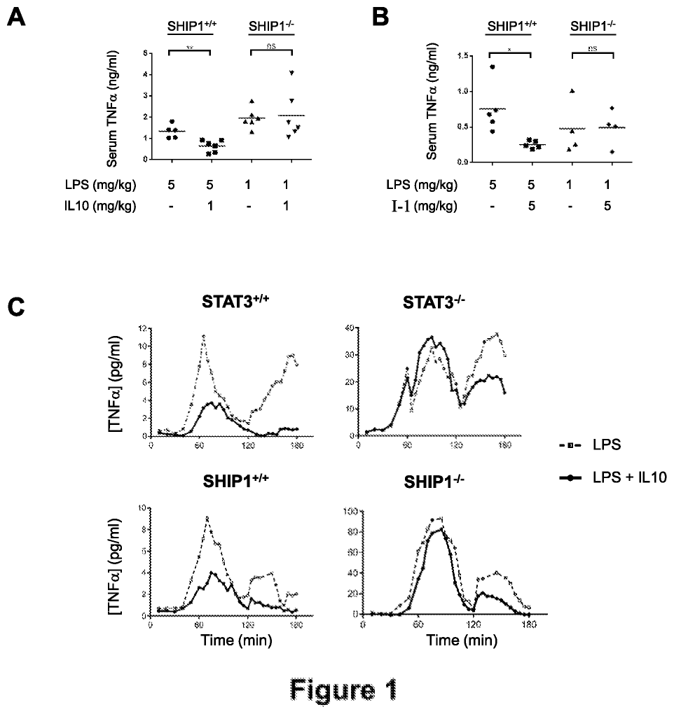

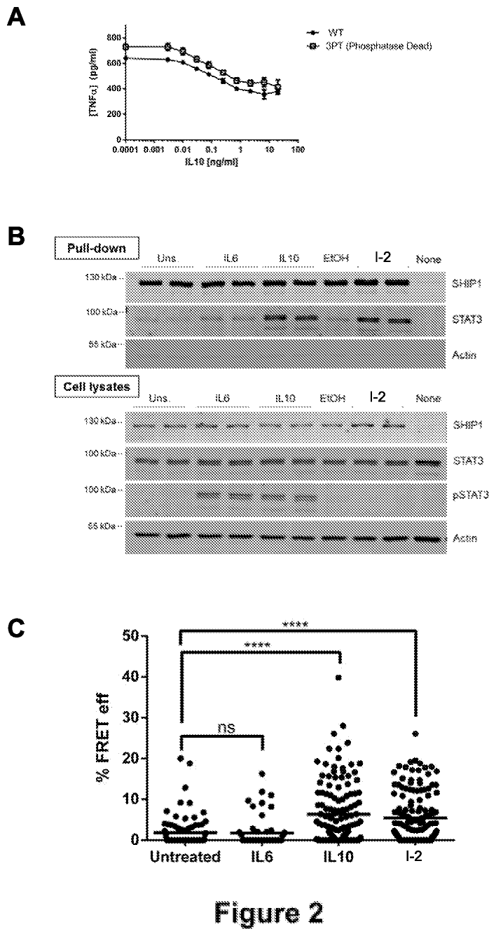

A role for STAT3 in mediating IL10 inhibition of TNFα in vivo was first described by Takeda et al (Takeda et al., 1999). It was found that LPS administration to mice with a myeloid specific knockdown of STAT3 produced more TNFα than wild-type mice, concluding endogenous IL10 is unable to counteract LPS signaling in the STAT3^{-/-} mice. However, closer examination of their data showed that while TNFα levels remain high post LPS administration in the IL10^{-/-} mice (Berg et al., 1995), TNFα levels drop in STAT3^{-/-} mice as endogenous levels of IL10 rise (Takeda et al, FIG. 2B). This implies that a protein other than STAT3 might contribute to IL10 action. It has been shown previously that culture-based studies suggested that SHIP1 participates in IL10 action (Chan et al., 2012, Cheung et al., 2013). Presently, the ability of IL10 to inhibit LPS-induced inflammatory cytokine/chemokine expression in vivo is shown (FIG. 1A) in SHIP1^{+/+} and SHIP1^{-/-} mice, and found IL10 inhibited TNFα in SHIP1^{+/+} but not SHIP1^{-/-} mice. It was previously shown that SHIP1 phosphatase activity is allosterically stimulated by its product PI(3,4)P2 (Ong et al., 2007). Compound I-1 (previously called AQX-MN100) binds to the same SHIP1 C2 domain as PI(3,4)P2 and increases SHIP1 functional activity (Ong et al., 2007). Presently, it is shown that compound I-1 inhibits LPS-induced TNFα in SHIP1^{+/+} but not SHIP1^{-/-} mice, showing that these compounds can mimic anti-inflammatory properties of IL10 and is indeed specific for SHIP1 (FIG. 1B).

Without wishing to be bound by theory, SHIP1 and STAT3 could be acting independently or together in mediating IL10 action. To help distinguish between these two possibilities a continuous flow cell culture system was used to assess the kinetics of TNFα production in SHIP1 and STAT3 wild-type and knockout bone marrow derived macrophages (BMDM). LPS stimulates two peaks of TNFα expression, one at around 1 hour and another at 3 hours (FIG. 1C). IL10 reduces TNFα levels in both SHIP1^{+/+} and STAT3^{+/+} cells, but is completely impaired in inhibiting the 1-hour peak in both STAT3^{-/-} and SHIP1^{-/-} cells, and partly impaired in inhibiting the 3-hour peak in both KO BMDM. The identical patterns of non-responsiveness suggest that SHIP1 and STAT3 cooperate.

FIG. 1 shows serum TNFα level of SHIP1^{+/+} or SHIP1^{-/-} mice injected intra-peritoneally with LPS, LPS+IL10 (Panel A), or LPS+compound I-1 (ZPR-100, or ZPR-MN100, or MN-100) (Panel B) at the concentrations indicated for 1 h. Data represent means of *p<0.05, **p<0.01 when compared with LPS-alone-stimulated mice, ns=not significant. Panel C shows STAT3^{+/+}, STAT^{-/-}, SHIP1^{+/+}, and SHIP1^{-/-} bone marrow-derived macrophages (BMDM) were stimulated with LPS (dotted line) or LPS+IL10 (solid line) over the course of 180 min in a continuous-flow apparatus. Fractions were collected every 5 min for measurement of TNFα levels Data are representative of two independent experiments.

### Example 2 IL10 Induces Physical Association of SHIP1 and STAT3 in Macrophages

Both SHIP1 and STAT3 proteins reside in the cytoplasm in resting cells and are recruited to the cell membrane in response to extracellular stimuli but through distinct mechanisms. STAT3 functions mostly as a transcription factor (Matsuda et al., 2015) and SHIP1 is best known for its lipid phosphatase activity (Pauls and Marshall, 2017). However, SHIP1 can also act as a docking or adaptor protein for assembly of signaling complexes (Pauls and Marshall, 2017). Indeed, a version of SHIP1 with minimal phosphatase activity (3PT) (An et al., 2005) was found to mediate the inhibitory effect of IL10 on LPS-stimulated TNFα production (FIG. 2A), it was then examined whether SHIP1 might serve an adaptor function in IL10 signaling and associate with STAT3 in response to IL10. FIG. 2B shows that treatment of cells with IL10 resulted in co-precipitation of SHIP1 with STAT3. IL6 failed to induce STAT3 association with SHIP1, even though STAT3 becomes tyrosine phosphorylated to the same extent as in response to IL10. Remarkably, treatment of cells with the small molecule SHIP1 allosteric regulator compound I-2, is sufficient to induce association of SHIP1 and STAT3 (FIG. 2B). The ability of compound I-2 to induce association of SHIP1 and STAT3 suggests the binding of compound I-2 may induce a conformational change that can alter SHIP1's association with other proteins. To see if the SHIP1/STAT3 interaction occurs in intact cells, Clover-SHIP1 and mRuby2-STAT3 fusion protein constructs were generated and transduced them into J17 SHIP1^{-/-} cells for FRET analysis. FIG. 2C shows that stimulating Clover-SHIP1/mRuby2-STAT3 cells with IL10 or compound I-2, but not IL6, increases the Clover-mRuby2 FRET signal suggesting SHIP1 and STAT3 interact in vivo.

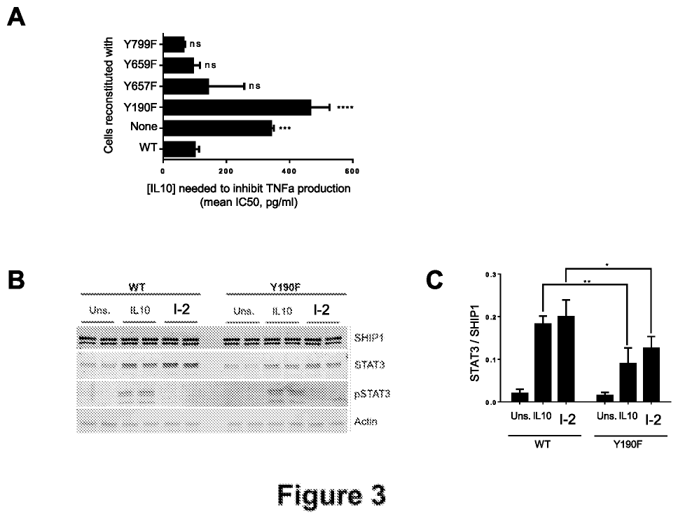

Both SHIP1 and STAT3 have SH2 domains and both have been reported to become phosphorylated on tyrosine residues, so the complex formation might be mediated through a phospho-tyrosine/SH2 interaction. Since FIG. 2B shows that STAT3 does not have to be phosphorylated to bind to SHIP1 (see I-2 lane), whether tyrosine residues on SHIP1 might become phosphorylated to interact with the STAT3 SH2 domain was looked at. Four tyrosine residues in SHIP1 exist in the context of a STAT3 SH2 domain recognition sequence. SHIP1 mutants were constructed in which each of these residues are substituted with phenylalanine, expressed them in the J17 SHIP1^{-/-} macrophage cell line and tested the ability of IL10 to inhibit TNFα expression (FIG. 3A) in these cells. Cells expressing the Y190F mutant behaved like a SHIP1^{-/-} (FIG. 3A) cell. The Y190F mutant ability to interact with STAT3 was reduced 2 fold in response to IL10 and compound I-2 (FIGS. 3B and 3C), suggesting that part of the SHIP1 interaction with STAT3 required phosphorylation of SHIP1 Y190.

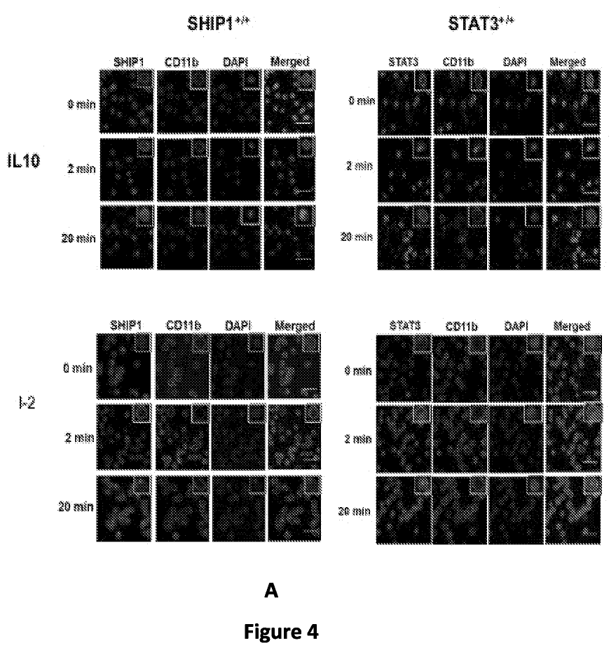

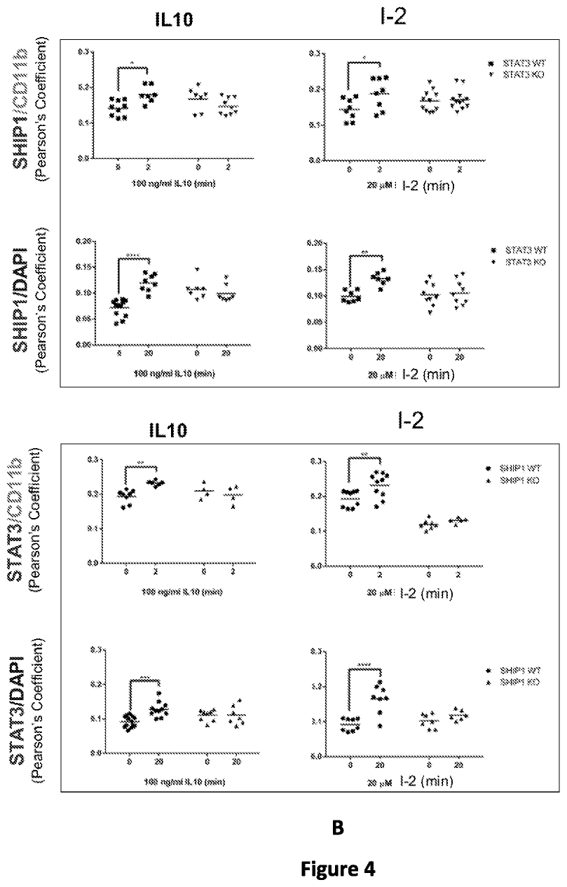

The subcellular localization of SHIP1 and STAT3 in primary cells was also assessed. Wild-type, SHIP1^{-/-} or STAT3^{-/-} peritoneal macrophages were stimulated with IL10 or compound I-2 and stained with antibodies against SHIP1 or STAT3. FIG. 4A and FIG. 4B shows IL10 or compound I-2 induced membrane association of both SHIP1 and STAT3 at 2 min in wild-type cells. SHIP1 does not translocate in STAT3_{-/-}cells, and STAT3 does not translocate in SHIP1_{-/-}cells (FIG. 4B). At 20 min, both SHIP1 and STAT3 are found in the nucleus in wild-type cells, and translocation required cells to express both STAT3 and SHIP1. Thus, compound I-1 can mimic IL10 in with respect to SHIP1 and STAT3 translocations.

### Example 3 SHIP1 Undergoes a Conformational Change Upon Allosteric Regulator Binding

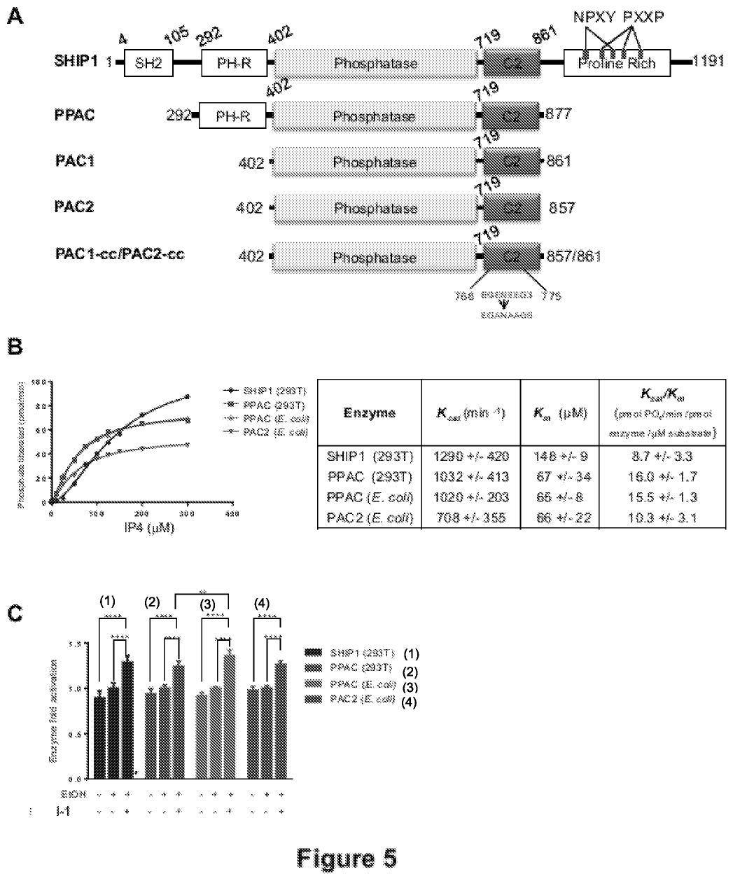

To better understand the interaction of the small molecule allosteric regulators with SHIP1, truncated SHIP1 proteins which contains the minimal region of SHIP1 needed for allosteric regulated phosphatase activity were produced for X-ray crystallography (FIG. 5). Full length SHIP1 cannot be expressed at sufficient quantity for crystallography so the minimal region of SHIP1 needed for allosteric activation was defined. It was previously shown the C2 domain binds the SHIP1 allosteric regulators (PI(3,4)P2, compound I-1), and that the PH-R domain N-terminal to the phosphatase domain might be involved (Ong et al., 2007). Full length SHIP1 (which could only be produced in mammalian 293T cells), PPAC (which contains the PH-R-phosphatase-C2 domains, and can be expressed in both 293T cells and E. coli), and PAC1/PAC2 (which contain the phosphatase-C2 domains, and can be expressed in E. coli) proteins (FIG. 5A) were expressed. Their enzyme (phosphatase) kinetic properties and ability to become activated by compound I-1 were examined. It was found that 293T and E. coli derived PPAC had the same enzymatic properties (FIG. 5B), and that all four proteins (full length SHIP1, 293T derived PPAC, E. coli derived PPAC, and PAC2) could be activated by compound I-1 (FIG. 5C).

Only PAC1 and PAC2 proteins could be expressed in amounts needed for structural studies so these were produced and put through screens for conditions to produce crystals of sufficient quality for structural determination. This included making surface entropy reduced (Derewenda, 2004, Goldschmidt et al., 2007) versions called PAC1-cc and PAC2-cc where three glutamic acid residues in PAC1 and PAC2 were substituted with alanines. The structure for several PAC1-cc and PAC2-cc crystals have been solved and is available in the PDB (PDB ID 6DLG), and the data from a PAC2-cc crystal which diffracted at 1.6 Å resolution is shown in Table 1. Small angle X-ray scattering data (SAXS) has been used to generate models of PAC1 with and without compound I-1. The solution conformation of unliganded PAC1 determined by SAXS confirms the X-ray crystal structure.

TABLE 1
Data of PAC2-cc Crystal

| Protein sample | SHIP1 PAC2-cc | |
|---|---|---|
| PDB ID | 6DLG | |
| Data collection | APS 23ID-D | |
| Wavelength (Å) | 1.03319 | |
| Space group | P 21 21 21 | |
| Unit cell parameters (Å) | a = 45.10, b = 73.20, c = 124.21 | |
| Unit cell angles (°) | α = 90.0, β = 90.0 γ = 90.0 | |
| Resolution (Å)* | 47.36-1.50 | (1.59-1.50) |
| Rₘₑₐₛ (%) | 5.9 | (78.7) |
| CC_{1/2} (%) | 99.9 | (68.2) |
| I/σ(I) | 13.79 | (1.85) |
| Completeness (%) | 98.9 | (98.6) |
| No. unique reflections | 66070 | (10528) |
| Redundancy | 3.28 | (3.29) |

| | |
|---|---|
| Wilson B factor (Å²) | 25.38 |
| Refinement | Phenix 1.13-2998 |
| Resolution (Å) | 38.4-1.50 |
| Solvent content (%) | 38 |
| R_{work}/R_{free} (%) | 17.85/20.04 |
| Ramachandran plot | 99.3/0.2 |
| favoured/outlier (%) | |
| No. of atoms | |
| Protein | 3628 |
| Isopropanol | 4 |
| Water | 246 |
| B-factors (Å²) | |
| Protein | 24 |
| Isopropanol | 26.6 |
| Water | 30.9 |
| RMSD bonds lengths (Å) | 0.008 |
| RMSD bond angles (°) | 0.966 |

*Values of the highest resolution shell are listed within parentheses

Furthermore, SAXS analysis showed that the binding of compound I-1 to PAC1 results in a change in its overall conformation.

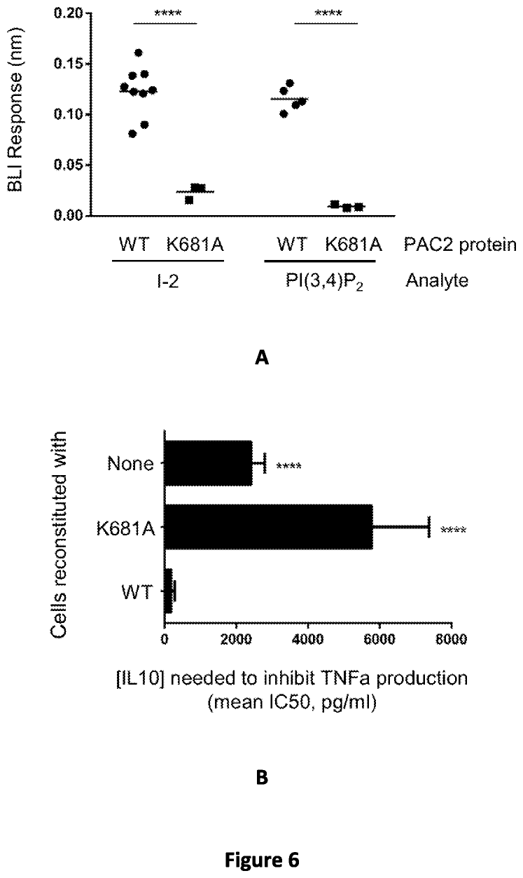

Using molecular modeling (Fuqiang et al., 2018) it is identified a potential binding pocket for compound I-1 and I-2 in PAC2. Residue K681 in this pocket is predicted to be involved in binding compound I-1 and I-2 so the K681A point mutant of PAC2 was generated and tested for the ability of wild-type and PAC2-K681A to bind compound I-2 using Biolayer Interferometry (BLI). As shown in FIG. 6A, substitution of K681A in the putative pocket impairs the ability of both compound I-2 and PI(3,4)P2 to bind to PAC2. The effect of the K681A substitution on the ability of full length SHIP1 to mediate IL10 inhibition of macrophages was assessed. FIG. 6B shows that IL10 inhibited TNFα production efficiently in cells expressing wild-type but not K681A SHIP1.

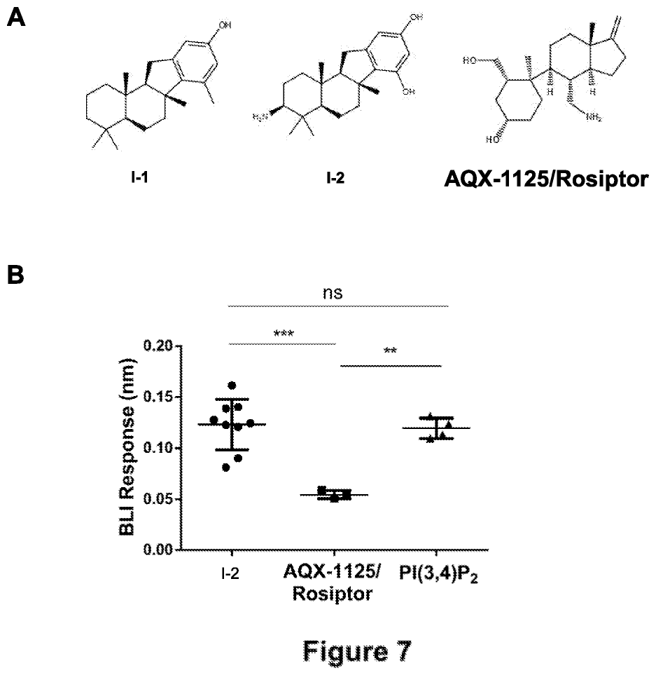

Stenton et al described a molecule called AQX-1125 (structure in FIG. 7A, later given the clinical trial name of Rosiptor) as a SHIP1 agonist (Stenton et al., 2013a, Stenton et al., 2013b). However AQX-1125/Rosiptor has marginal SHIP1 phosphatase enhancing activity (Stenton et al., 2013b), and displayed different enzyme kinetics properties (Stenton et al., 2013b) than observed with compound I-1 (Ong et al., 2007). Stenton et al looked at binding of tritiated AQX-1125/Rosiptor to SHIP1 protein using scintillation proximity assay but it is difficult to assess the significance of the ˜300 cpm signal they observed. So here the ability of PI(3,4)P2, compound I-2 and AQX-1125/Rosiptor to bind to SHIP1 was compared in BLI assay (FIG. 7B). It was found AQX-1125/Rosiptor binds very poorly to SHIP1 as compared to compound I-2 or SHIP1's natural agonist PI(3,4)P2.

### Example 4 Alleviation of Inflammation in IL-10^{-/-} Mouse Model Colitis

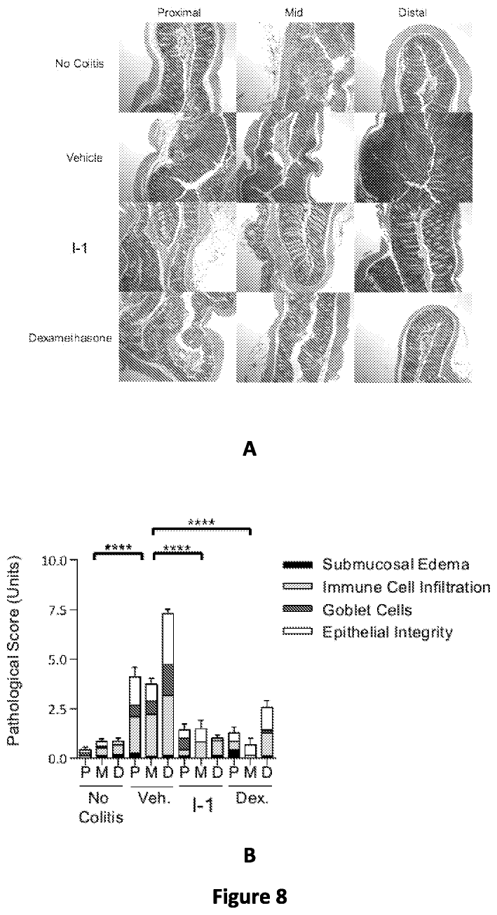

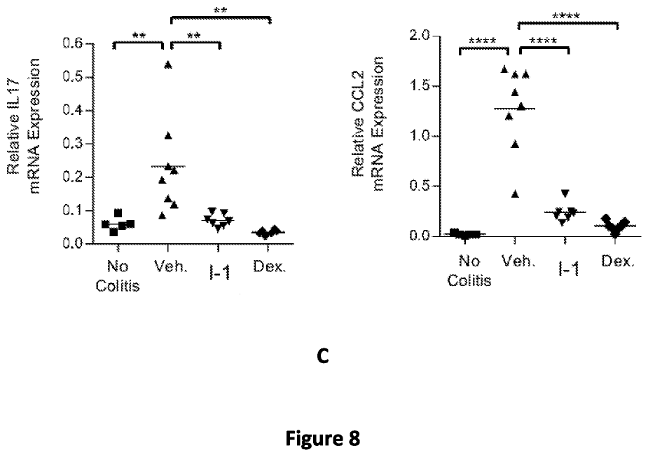

To show in vivo anti-inflammatory effect of the compounds of the present disclosure, exemplary compound I-1 was administered to IL-10 knock-out mouse model of colitis (Keubler 2015). IL10 knock-out mice develop colitis when colonized with normal gut flora because IL10 is needed to temper the host immune response to intestinal commensal bacteria (Keubler 2015, Kuhn 1993). Colitis was initiated in IL10−/−mice by inoculating them with the freshly isolated colon contents of normal, specific pathogen free mice and allowed inflammation to develop for 6 weeks (Sydora 2003). Mice were then treated for 3 weeks with vehicle, 2 mg/kg compound I-1, or 0.4 mg/kg dexamethasone (anti-inflammatory steroidal drug used as positive control) prior to colon tissue collection for analyses. Hematoxylin and eosin stained sections were prepared from the proximal, mid and distal colons of mice, as well as from mice not inoculated with flora (no colitis group) (FIG. 8A).

Two investigators blinded to the treatment groups scored the sections based on submucosal edema, immune cell infiltration, presence of goblet cells and epithelial integrity (FIG. 8B). In the three groups where colitis was induced, the dexamethasone and compound I-1 groups had significantly lower pathology scores than the vehicle group (FIG. 8B). RNA was prepared from the colons of all four groups for analysis of IL17 and CCL2 expression, inflammatory mediators elevated in colitis (Lee 2007). As shown in FIG. 8C, both compound I-1 and dexamethasone treatment significantly reduced the levels of IL17 and CCL2 mRNA. These data indicate that compound I-1 treatment can reduce inflammation in colitis resulting from the loss of MO. It was found that compound I-1 was as effective as dexamethasone in reducing histological and molecular markers of colon inflammation.

### Example 5 Discussion

IL6 and MO have opposing pro- and anti-inflammatory actions respectively on macrophages (Garbers et al., 2015, Yasukawa et al., 2003) but both cytokines stimulate tyrosine phosphorylation of STAT3 Y705 in cells. It was found that MO but not IL6 induced association of STAT3 with SHIP1, and suggest this difference may contribute to why STAT3 can mediate pro- and anti-inflammatory responses downstream of both cytokines. IL10-induced SHIP1/STAT3 signaling support anti-inflammatory responses while IL6-induced STAT3/STAT3 dimers support pro-inflammatory responses. Yasukawa et als study of SOCS3 knockout cells suggested that the duration of STAT3 activation in macrophage cells can underlie the opposite biological effects of IL10 and IL6 (Yasukawa et al., 2003). The present data are compatible with theirs since STAT3 activation can also be prolonged by its association with SHIP1.

The question of how STAT3 can mediate opposing biological responses has also been explored for opposing effects of IL6 vs IL27 on naïve T cell differentiation. Treatment of naïve T cells with IL6 inhibits Th1, and enhances Th2 and Th17 differentiation, while IL27 treatment does the opposite (Hirahara et al., 2015, Peters et al., 2015). Both IL6 and IL27 activate STAT3 equally strongly, but IL27 activates STAT1 more strongly than IL6. Hirahara et al used genomic approaches to conclude that STAT3 controls the overall magnitude of transcription but that the level of STAT1 activation governs the expression of IL27 specific gene expression (Hirahara et al., 2015). Peters et al reported observations consistent with this conclusion in that both IL6 and IL27 stimulate Th17 differentiation if STAT1 is deleted (Peters et al., 2015).

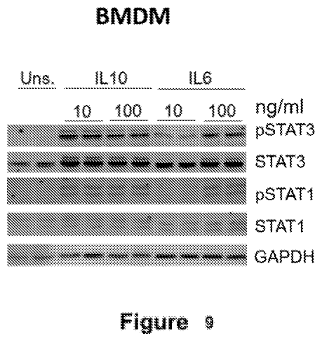

However, the opposing effect of MO and IL6 on macrophage activation is not due to differential use of STAT3 or STAT1 as both cytokines induce similar phosphorylation of STAT1 and STAT3 (FIG. 9).

It was previously showed that small molecule SHIP1 agonists have anti-inflammatory actions in vitro (Meimetis et al., 2012, Ong et al., 2007) and ascribed these actions to the stimulation of SHIP1's phosphatase to dephosphorylate the PI3K product PIP3 into PI(3,4)P2 (Fernandes et al., 2013, Huber et al., 1999, Krystal, 2000, Pauls and Marshall, 2017). However, the present data demonstrate a SHIP1 protein with non-detectable phosphatase activity is sufficient to mediate the anti-inflammatory effect of MO, so the adaptor function of SHIP1 can by itself support MO action. The present SAXS analyses suggest that the binding of SHIP1 agonists to SHIP1 causes a conformational change in SHIP1. This conformational change may allow SHIP1 to interact with STAT3 and the complex of SHIP1/STAT3 to translocate to the nucleus. The solved structure of the minimal domain (PAC1/2) of SHIP1 required to mediate the allosteric action of SHIP1 agonists, and identified a drug binding pocket through molecular docking analysis. Mutation of a residue predicted to be involved in binding to compound I-2 abolished compound I-2 binding and the ability of SHIP1 to mediate MO inhibition of TNFα expression in macrophages.

It was found that SHIP1 Y190 contributes to the ability of SHIP1 to associate with STAT3. The Y190F mutant's ability to interact with STAT3 was reduced 2 fold as compared to wild-type SHIP1 (FIG. 3B). However SHIP1 Y190F is completely impaired in its ability to support IL10 inhibition of TNFα (FIG. 3A). Without wishing to be bound by theory, one interpretation is the partial SHIP1/STAT3 complex inhibition is physiologically significant because inhibition of TNFα is completely abolished. Alternatively, the SHIP1/STAT3 complex formation is only one function of the Y190. The SHIP1 agonist compound I-1 could by itself induce formation of a SHIP1/STAT3 complex. The addition of compound I-2 to perimacs could also induce translocation of SHIP1 and STAT3 to the nucleus. Together these data suggest that the action of SHIP1 agonists includes both their ability to stimulate SHIP1 phosphatase activity and to induce the association of SHIP1 with STAT3.

Treatment of macrophages with compound I-1 or I-2 was sufficient to elicit the anti-inflammatory effects similar to that of IL10 in vitro in this and previous studies (Chan et al., 2012, Cheung et al., 2013, Ong et al., 2007). Thus, compound I-1 was tested in a mouse model of colitis since the beneficial anti-inflammatory action of IL10 in colitis is through IL10 action on macrophages (Friedrich et al., 2019, Ouyang and O'Garra, 2019, Shouval et al., 2014b, Zigmond et al., 2014). It was found that compound I-1 was as effective as dexamethasone in reducing histological and molecular markers of colon inflammation. Medzhitov's group recently reported IL10 stimulation of mitophagy and inactivation of the inflammasome as part of its protective effect in colitis, and that this involved STAT3-dependent upregulation of the DDIT4 protein (Ip et al., 2017). It has been confirmed here IL10 upregulation of DDIT4 in macrophages requires both STAT3 and SHIP1; furthermore, compound I-2 was by itself able to induce DDIT4 expression.

A small molecule SHIP1 allosteric regulator (AQX-1125/Rosiptor) (Stenton et al., 2013a, Stenton et al., 2013b) was recently tested in clinical trials for relief of urinary bladder pain experienced by interstitial cystitis (IC) patients (Nickel et al., 2016). IC reportedly was chosen for the disease indication because: AQX-1125/Rosiptor accumulates in the urinary bladder (Stenton et al., 2013b), two papers implicated PI3K-dependent inflammation in IC (Liang et al., 2016, Qiao et al., 2014), and preliminary phase 2 trials seemed promising (Nickel et al., 2016). However, the phase 3 trial failed to show efficacy for AQX-1125/Rosiptor (AQXP, 2018). There are many reasons for small molecule drugs to fail during the drug development process. However, it is noted that neither IL10 nor SHIP1 has been implicated in the physiology/pathophysiology of IC. Furthermore, it was found that AQX-1125/Rosiptor has very weak binding to SHIP1, consistent with Stenton's et al finding that AQX-1125 has very weak SHIP1 phosphatase activating ability (Stenton et al., 2013b).

In light of the above, it can be seen that disease indications for which small molecule SHIP1 allosteric regulators are developed should be ones in which IL10 (or other physiological regulators of SHIP1) (Chan et al., 2012, Cheung et al., 2013, Dobranowski and Sly, 2018, Pauls and Marshall, 2017) has been shown to play a beneficial role. These small molecules should also have similar binding properties to SHIP1 as its natural ligand PI(3,4)P2 By these criteria, small molecule SHIP1 agonists such as those of the Pelorol family can be used for the treatment of human inflammatory bowel disease.

### Example 6 Inhibition of MM in Mouse Model

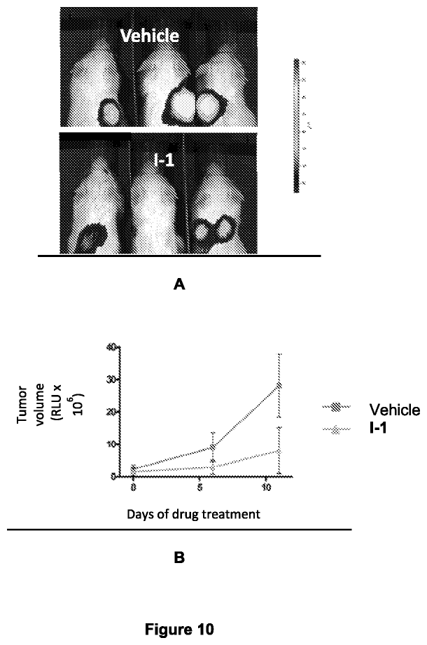

MM.1S cells expressing firefly luciferase were injected along with Matrigel basement membrane into the upper flank of NOD/SCID mice and allowed to establish for two weeks. Compound I-1 or vehicle (n=4) were administered through drinking water (as described in General Methods). Bioluminescence images of control and Compound I-1 treated mice were taken and shown in FIG. 10A. Tumour volume was quantified using bioluminescence imaging and shown in FIG. 10B. Administration of compound I-1 to mice bearing MM tumours effectively reduces tumour mass.

### Example 7 Compound Efficacy in Protection of Liver Injury

Concanavalin A (ConA) induced liver injury model is an immune-mediated liver injury model, resembling viral and autoimmune hepatitis in humans. Intravenous delivery of ConA in mice is known to activate T cells, resulting in increased inflammatory cytokines such as TNF-α, IFN-r and IL-6 as well as decreased anti-inflammatory cytokine IL-10. The T cell infiltration into the liver leads to consequences of hepatocyte apoptosis and necrosis, resulting in increased levels of liver enzymes ALT and AST in plasma. (Zhou 2015)

Methods: Five groups (n=5 each) of C57 mice were treated with a blank control, compound I-1 alone (10 mg/kg/d), ConA (15 mg/kg), ConA+compound I-1 (3 mg/kg/d) or ConA+compound I-1 (10 mg/kg/d). In each instance, compound I-1 was given 5 days before ConA injection (if applicable) via oral gavage twice a day. One hour after the last drug administration, ConA (15 mg/kg) was delivered intravenously. 12 hours after the ConA injection, orbital blood was taken from the eye and the enzyme biochemistry (plasma) and blood cell type tests were performed by analyzers.

Results: As shown in panels A to D of FIG. 11, ConA at 15 mg/kg significantly increased the levels of ALT, AST, TBIL and BUN (FIG. 11A to D). Compound I-1 (MN-100) significantly reduced the levels of ALT, AST, TBIL and BUN (*P<0.05), demonstrating anti-inflammatory effects in ConA-induced liver injury. ALT and AST plasma levels were determined in a repeat experiment, where dexamethasone (0.5 mg/kg/d) was administered with ConA as a positive control. (Panels E and F of FIG. 11). It can be seen that compound I-1 significantly lowered ConA-induced ALT and AST levels similarly to the positive control dexamethasone.

### Example 8 SHIP1 Activator for Treatment of Severe Sepsis in a Mouse Caecum Ligation and Puncture (CLP) Model

The mouse CLP model is well-accepted clinically relevant method for anti-sepsis drug testing. The surgical operation included opening of the mouse abdomen, ligation of the cecum and puncture of the ligated cecum with a needle. Rittirsch, 2009 Method was followed to induce Mid to High grade experimental sepsis to achieve 70-100% survival rate over 7 days after CLP operation.

Methods: Five groups (n=10 each) of mice were designed with (1) a blank control, (2) Sham control (abdomen operation was performed but without cecal ligation/puncture), (3) CLP group (operation with cecal ligation/puncture), (4) CLP+compound I-1 (MN100) (3 mg/kg/d), and (5) CLP+compound I-1 (MN100) (10 mg/kg/d). ZPR-MN100 (compound I-1) was given 3 days before CLP operation and continued to 7 days after CLP. ZPR-MN100 was delivered via oral gavage twice a day. 24 hours after CLP, tail blood was taken under sterile environment for blood culture to demonstrate septic infection after CLP operation. Mouse condition and survival were recorded every day. Seven days after CLP, the experiment was terminated and survival curves were drawn using GraphPad Prism.

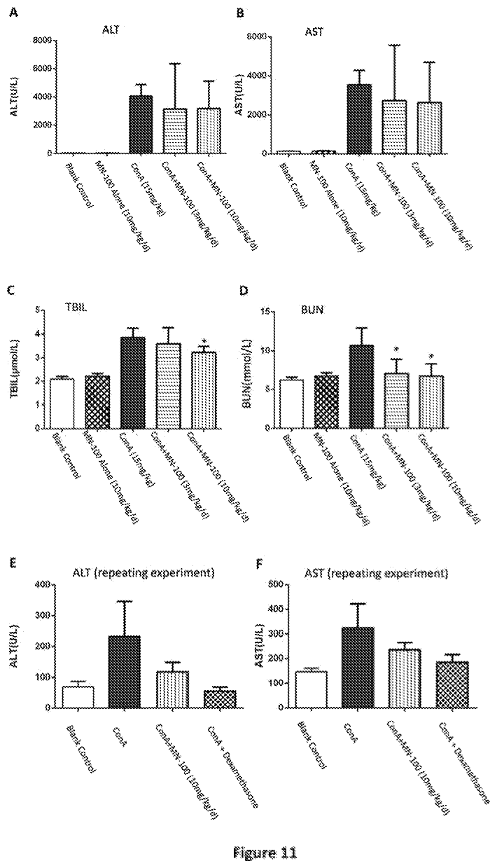

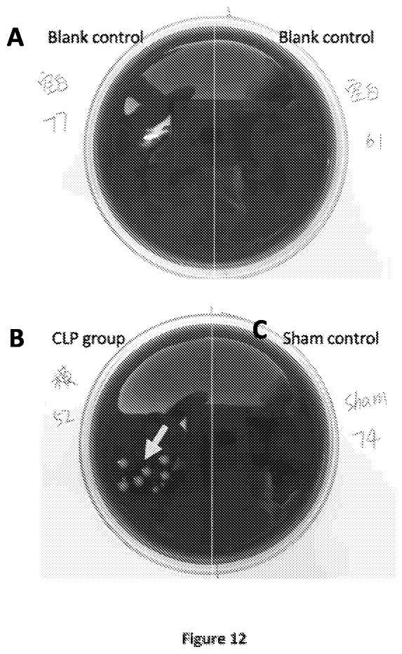

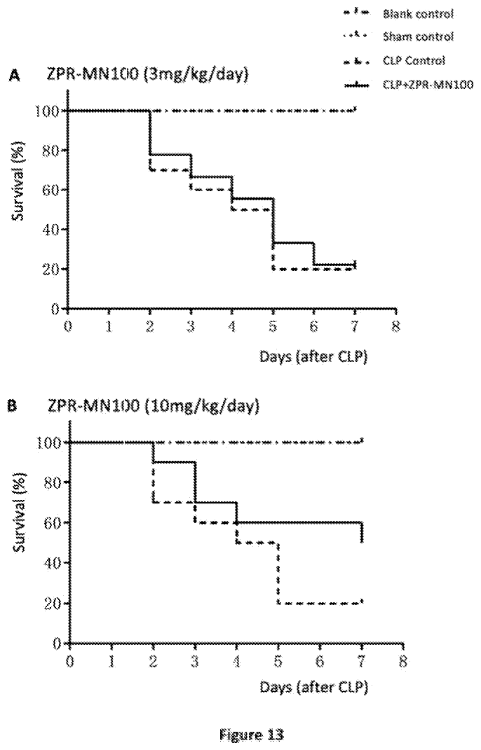

Results: As shown in FIG. 12, twenty-four hours after CLP operation, bacteria colonies were formed from the blood culture of the CLP operation group (FIG. 12B) but not from blank control (FIG. 12A) or sham control (FIG. 12C, the mice only received skin incision and wound closure but without cecum ligation or puncture), demonstrating successful establishment of the CLP model. The oral delivery of compound 1-1 dose-dependently protected the mice from CLP-induced mortality (FIG. 13). The CLP group without compound I-1 treatment has a 7-day survival rate of 20%. (FIG. 13A) However, compound I-1 was able to increase the survival rate in a dose-dependent manner to 50% mice alive (vs. 20% survival rate in CLP group without drug).

### Example 9 Stimulating IL-10/IL-1 OR Pathway as Treatment for Allergy and Asthma

It has been established that IL-10 plays a very important role in inhibition of the allergic inflammation and protect the development of allergic airway diseases and asthma (Hawrylowicz et al, 2005; Coomes S M et al, 2015). Allergic rhinitis (AR) is a prevalent inflammatory airway disease without an effective treatment. In mouse model, recombinant IL-10 administration in OVA-induced AR model appeared to reduce the number of eosinophils and mast cells in nasal mucosa in the AR mice (Wang et al, 2014), suggesting IL10/IL10R pathway as a valid target for AR treatment. Small molecules SHIP1 agonists such as compound I-2 (ZPR-151) can be useful to inhibit nasal inflammatory response by activating IL-10/IL10R pathway.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### FULL CITATIONS OF DOCUMENTS REFERRED IN THE PRESENT DISCLOSURE

Abramson, H. N. The Multiple Myeloma Drug Pipeline-2018: A Review of Small Molecules and Their Therapeutic Targets. Clinical Lymphoma Myeloma and Leukemia 18, 611-627 (2018).
Aiken, S. & Izban, K. F. Immunohistochemical localization of phosphorylated AKT in multiple myeloma. Blood 99, 2278-2279 (2002).
Aman, M. J., Lamkin, T. D., Okada, H., Kurosaki, T. & Ravichandran, K. S. The inositol phosphatase SHIP inhibits Akt/PKB activation in B cells. The Journal of biological chemistry 273, 33922-33928 (1998).
An, H., Xu, H., Zhang, M., Zhou, J., Feng, T., Qian, C., Qi, R. & Cao, X. 2005. Src homology 2 domain-containing inositol-5-phosphatase 1 (SHIP1) negatively regulates TLR4-mediated LPS response primarily through a phosphatase activity- and PI-3K-independent mechanism. Blood 105, 4685-92.
Asadullah K, Sterry W, Volk H D. Interleukin-10 therapy—review of a new approach. Pharmacological Reviews. 2003; 55(2):241-269.
AQXP. 2018. Aquinox Announces Topline Results of Phase 3 LEADERSHIP 301 Clinical Trial in Interstitial Cystitis/Bladder Pain Syndrome http://investoraqxpharma.com/news-releases/news-release-details/aquinox-announces-topline-results-phase-3-leadership-301 [Online]. Available: http://investor.aqxpharma.com/news-releases/news-release-details/aquinox-announces-topline-results-phase-3-leadership-301 [Accessed].
Berg, D. J., Kuhn, R., Rajewsky, K., Muller, W., Menon, S., Davidson, N., Grunig, G. & Rennick, D. 1995. Interleukin-10 is a central regulator of the response to LPS in murine models of endotoxic shock and the Shwartzman reaction but not endotoxin tolerance. J Clin Invest 96, 2339-47.
Benard E L, van der Sar A M, Ellett F, Lieschke G J, Spaink H P, Meijer A H. Infection of zebrafish embryos with intracellular bacterial pathogens. J Vis Exp. 2012 Mar. 15; (61). pii: 3781. doi: 10.3791/3781. PubMed PMID: 22453760; PubMed Central PMCID: PMC3415172.
Brown, J., Wang, H., Hajishengallis, G. N. & Martin, M. TLR-signaling Networks: An Integration of Adaptor Molecules, Kinases, and Cross-talk. Journal of Dental Research 90, 417-427 (2010).
Chan, C. S., Ming-Lum, A., Golds, G. B., Lee, S. J., Anderson, R. J. & Mui, A. L. 2012. Interleukin-10 inhibits lipopolysaccharide-induced tumor necrosis factor-alpha translation through a SHIP1-dependent pathway. J Biol Chem 287, 38020-7.
Cheung, S. T., So, E. Y., Chang, D., Ming-Lum, A. & Mui, A. L. Interleukin-10 inhibits lipopolysaccharide induced miR-155 precursor stability and maturation. PloS one 8, e71336 (2013).
Coomes S M, Kannan Y, Pelly V S, Entwistle L J, Guidi R, Perez-Lloret J, Nikolov N, Muller W, Wilson M S. CD4(+) Th2 cells are directly regulated by IL-10 during allergic airway inflammation. Mucosal Immunol. 2017 January; 10(1):150-161. doi: 10.1038/mi.2016.47. Epub 2016 May 11. PubMed PMID: 27166557.
Czaja A J. Immune Inhibitory Properties and Therapeutic Prospects of Transforming Growth Factor-Beta and Interleukin 10 in Autoimmune Hepatitis. Dig Dis Sci. 2021 Apr. 9. doi: 10.1007/s10620-021-06968-6. Online ahead of print.
Derewenda, Z. S. 2004. Rational protein crystallization by mutational surface engineering. Structure 12, 529-35.
Dobranowski, P. & Sly, L. M. 2018. SHIP negatively regulates type II immune responses in mast cells and macrophages. J Leukoc Biol.
Engelhardt, K. R. & Grimbacher, B. IL-10 in humans: lessons from the gut, IL-10/IL-10 receptor deficiencies, and IL-10 polymorphisms. Current topics in microbiology and immunology 380, 1-18 (2014).
Fernandes, S., Iyer, S. & Kerr, W. G. Role of SHIP1 in cancer and mucosal inflammation. Annals of the New York Academy of Sciences 1280, 6-10 (2013).
Fleming A, Jankowski J, Goldsmith P. In vivo analysis of gut function and disease changes in a zebrafish larvae model of inflammatory bowel disease: a feasibility study. Inflamm Bowel Dis. 2010 July; 16(7):1162-72. doi: 10.1002/ibd.21200. PubMed PMID: 20128011.
Friedrich, M., Pohin, M. & Powrie, F. 2019. Cytokine Networks in the Pathophysiology of Inflammatory Bowel Disease. Immunity 50, 992-1006.
Frost, P., et al. In vivo antitumor effects of the mTOR inhibitor CCI-779 against human multiple myeloma cells in a xenograft model. Blood 104, 4181-4187 (2004).
Fukuda, R., et al. Alteration of phosphatidylinositol 3-kinase cascade in the multilobulated nuclear formation of adult T cell leukemia/lymphoma (ATLL). Proc Natl Acad Sci USA 102, 15213-15218 (2005).
Fuqiang, B., Kush, D., Eric, L., Helene, M., S., R. P. & Artem, C. 2018. Cheminformatics Driven Development of Novel Therapies for Drug Resistant Prostate Cancer. Molecular Informatics 37, e1800043.
Garbers, C., Aparicio-Siegmund, S. & Rose-John, S. 2015. The IL-6/gp130/STAT3 signaling axis: recent advances towards specific inhibition. Current Opinion in Immunology 34, 75-82.
Ge, N. L. & Rudikoff, S. Expression of PTEN in PTEN-deficient multiple myeloma cells abolishes tumor growth in vivo. Oncogene 19, 4091-4095 (2000).
Glocker, E. O., et al. Inflammatory bowel disease and mutations affecting the interleukin-10 receptor. N Engl J Med 361, 2033-2045 (2009).
Glocker, E. O., Kotlarz, D., Klein, C., Shah, N. & Grimbacher, B. IL-10 and IL-10 receptor defects in humans. Annals of the New York Academy of Sciences 1246, 102-107 (2011).
Goldschmidt, L., Cooper, D. R., Derewenda, Z. S. & Eisenberg, D. 2007. Toward rational protein crystallization: A Web server for the design of crystallizable protein variants. Protein science: a publication of the Protein Society 16, 1569-76.
Harding, T., Baughn, L., Kumar, S. & Van Ness, B. The future of myeloma precision medicine: integrating the compendium of known drug resistance mechanisms with emerging tumor profiling technologies. Leukemia (2019).
Hawrylowicz C M. Regulatory T cells and IL-10 in allergic inflammation. J Exp Med. 2005 Dec. 5; 202(11):1459-63. Review. PubMed PMID: 16330811; PubMed Central PMCID: PMC2213335.
Helgason, C. D., et al. A dual role for Src homology 2 domain-containing inositol-5-phosphatase (SHIP) in immunity: aberrant development and enhanced function of b lymphocytes in ship −/− mice. J Exp Med 191, 781-794 (2000).
Hideshima, T., Nakamura, N., Chauhan, D. & Anderson, K. C. Biologic sequelae of interleukin-6 induced PI3-K/Akt signaling in multiple myeloma. Oncogene 20, 5991-6000 (2001).
Hideshima, T., et al. Perifosine, an oral bioactive novel alkylphospholipid, inhibits Akt and induces in vitro and in vivo cytotoxicity in human multiple myeloma cells. Blood 107, 4053-4062 (2006).
Hideshima, T., et al. Inhibition of Akt induces significant downregulation of survivin and cytotoxicity in human multiple myeloma cells. Br J Haematol 138, 783-791 (2007).
Hirahara, K., Onodera, A., Villarino, A. V., Bonelli, M., Sciume, G., Laurence, A., Sun, H. W., Brooks, S. R., Vahedi, G., Shih, H. Y., Gutierrez-Cruz, G., Iwata, S., Suzuki, R., Mikami, Y., Okamoto, Y.,
Nakayama, T., Holland, S. M., Hunter, C. A., Kanno, Y. & O'shea, J. J. 2015. Asymmetric Action of STAT Transcription Factors Drives Transcriptional Outputs and Cytokine Specificity. Immunity 42, 877-89.
Hsu, J., et al. The AKT kinase is activated in multiple myeloma tumor cells. Blood 98, 2853-2855 (2001).
Hu, L., et al. Downstream effectors of oncogenic ras in multiple myeloma cells. Blood 101, 3126-3135 (2003).
Hu, J. & Hu, W.-X. Targeting signaling pathways in multiple myeloma: Pathogenesis and implication for treatments. Cancer Letters 414, 214-221 (2018).
Huber, M., et al. The role of SHIP in growth factor induced signalling. Prog Biophys Mol Biol 71, 423-434 (1999).
Ip, W. K. E., Hoshi, N., Shouval, D. S., Snapper, S. & Medzhitov, R. 2017. Anti-inflammatory effect of IL-10 mediated by metabolic reprogramming of macrophages. Science (New York, N. Y 356, 513-519.
Kennah, M., et al. Activation of SHIP via a small molecule agonist kills multiple myeloma cells. Experimental Hematology 37, 1274-1283 (2009).
Keubler, L. M., Buettner, M., Hager, C. & Bleich, A. 2015. A Multihit Model: Colitis Lessons from the Interleukin-10-deficient Mouse. Inflammatory Bowel Diseases 21, 1967-1975.
Krystal, G. Lipid phosphatases in the immune system. Semin Immunol 12, 397-403 (2000).
Kuehl, W. M. & Bergsagel, P. L. Multiple myeloma: evolving genetic events and host interactions. Nature Reviews Cancer 2, 175 (2002).
Kuhn, R., Lohler, J., Rennick, D., Rajewsky, K. & Muller, W. Interleukin-10-deficient mice develop chronic enterocolitis. Cell 75, 263-274 (1993).
Lee, J. W., Bajwa, P. J., Carson, M. J., Jeske, D. R., Cong, Y., Elson, C. O., Lytle, C. & Straus, D. S. 2007. Fenofibrate represses interleukin-17 and interferon-gamma expression and improves colitis in interleukin-10-deficient mice. Gastroenterology 133, 108-23.
Li, J., et al. PTEN, a putative protein tyrosine phosphatase gene mutated in human brain, breast, and prostate cancer. Science 275, 1943-1947 (1997).
Li, D., et al. Turnagainolides A and B, Cyclic Depsipeptides Produced in Culture by a Bacillus sp.: Isolation, Structure Elucidation, and Synthesis. Journal of Natural Products 74, 1093-1099 (2011).
Liang, X., et al. Quantification of change in phosphorylation of BCR-ABL kinase and its substrates in response to Imatinib treatment in human chronic myelogenous leukemia cells. Proteomics 6, 4554-4564 (2006).
Liang, S., Li, J., Gou, X. & Chen, D. 2016. Blocking mammalian target of rapamycin alleviates bladder hyperactivity and pain in rats with cystitis. Molecular pain 12.
Liu, Q., et al. SHIP is a negative regulator of growth factor receptor-mediated PKB/Akt activation and myeloid cell survival. Genes Dev 13, 786-791 (1999).
Liu Q, Zhou Y H, Yang Z Q. (2016) The cytokine storm of severe influenza and development of immunomodulatory therapy. Cell Mol Immunol. 13(1):3-10.
Louis, E., Libioulle, C., Reenaers, C., Belaiche, J. & Georges, M. Genetics of ulcerative colitis: the come-back of interleukin 10. Gut 58, 1173-1176 (2009).
Louis H, Le Moine O, Goldman M, Devière J. Modulation of liver injury by interleukin-10. Acta Gastroenterol Belg. 2003 January-March; 66(1):7-14.
Luo, J. M., et al. Mutation analysis of SHIP gene in acute leukemia. Zhongguo Shi Yan Xue Ye Xue Za Zhi 12, 420-426 (2004).
Madsen, K., Cornish, A., Soper, P., Mckaigney, C., Jijon, H., Yachimec, C., Doyle, J., Jewell, L. & De Simone, C. 2001. Probiotic bacteria enhance murine and human intestinal epithelial barrier function. Gastroenterology 121, 580-91.
Marshall, J. C. 2014 Why have clinical trials in sepsis failed?. Trends in Mol. Med., 2014, 20:195-203.
Matsuda, T., Muromoto, R., Sekine, Y., Togi, S., Kitai, Y., Kon, S. & Oritani, K. 2015. Signal transducer and activator of transcription 3 regulation by novel binding partners. World journal of biological chemistry 6, 324-32.
Meimetis, L. G., Nodwell, M., Yang, L., Wang, X., Wu, J., Harwig, C., Stenton, G. R., Mackenzie, L. F., Macrury, T., Patrick, B. O., Ming-Lum, A., Ong, C. J., Krystal, G., Mui, A. L.-F. & Andersen, R. J. 2012. Synthesis of SHIP1-Activating Analogs of the Sponge Meroterpenoid Pelorol. European Journal of Organic Chemistry 2012, 5195-5207.
Ming-Lum, A., Shojania, S., So, E., Mccarrell, E., Shaw, E., Vu, D., Wang, I., Mcintosh, L. P. & Mui, A. L. 2012. A pleckstrin homology-related domain in SHIP1 mediates membrane localization during Fcgamma receptor-induced phagocytosis. FASEB J 26, 3163-77.
Mitsiades, C. S., et al. Activation of NF-kappaB and upregulation of intracellular anti-apoptotic proteins via the IGF-1/Akt signaling in human multiple myeloma cells: therapeutic implications. Oncogene 21, 5673-5683 (2002).
Miscevic F, Rotstein O and Wen X Y (2012) Advances in zebrafish high content and high throughput technologies, Combinatorial Chemistry and High Throughput Screening, 15:515-521
Naymagon, L. & Abdul-Hay, M. Novel agents in the treatment of multiple myeloma: a review about the future. Journal of Hematology & Oncology 9, 52 (2016).
Nickel, J. C., Egerdie, B., Davis, E., Evans, R., Mackenzie, L. & Shrewsbury, S. B. 2016. A Phase II Study of the Efficacy and Safety of the Novel Oral SHIP1 Activator AQX-1125 in Subjects with Moderate to Severe Interstitial Cystitis/Bladder Pain Syndrome. The Journal of urology 196, 747-754.
Patel, T. R., Meier, M., Li, J., Morris, G., Rowe, A. J. & Stetefeld, J. 2011. T-Shaped arrangement of the recombinant agrin G3-IgG Fc protein. Protein science: a publication of the Protein Society 20, 931-40.
Patel, T. R., Morris, G. A., Zwolanek, D., Keene, D. R., Li, J., Harding, S. E., Koch, M. & Stetefeld, J. 2010. Nano-structure of the laminin gamma-1 short arm reveals an extended and curved multidomain assembly. Matrix biology: journal of the International Society for Matrix Biology 29, 565-72.
Patel, T. R., Reuten, R., Xiong, S., Meier, M., Winzor, D. J., Koch, M. & Stetefeld, J. 2012. Determination of a molecular shape for netrin-4 from hydrodynamic and small angle X-ray scattering measurements. Matrix biology: journal of the International Society for Matrix Biology 31, 135-40.
Oehlers S H, Flores M V, Okuda K S, Hall C J, Crosier K E, Crosier P S. A chemical enterocolitis model in zebrafish larvae that is dependent on microbiota and responsive to pharmacological agents. Dev Dyn. 2011 January; 240(1):288-98. doi: 10.1002/dvdy.22519. PubMed PMID: 21181946.
Ong, C. J., Ming-Lum, A., Nodwell, M., Ghanipour, A., Yang, L., Williams, D. E., Kim, J., Demirjian, L., Qasimi, P., Ruschmann, J., Cao, L.-P., Ma, K., Chung, S. W., Duronio, V., Andersen, R. J.,
Krystal, G. & Mui, A. L.-F. 2007. Small-molecule agonists of SHIP1 inhibit the phosphoinositide 3-kinase pathway in hematopoietic cells. Blood 110, 1942-1949.
Ouyang, W., Rutz, S., Crellin, N. K., Valdez, P. A. & Hymowitz, S. G. Regulation and functions of the IL-10 family of cytokines in inflammation and disease. Annual review of immunology 29, 71-109 (2011).
Ouyang, W. & O'garra, A. 2019. IL-10 Family Cytokines IL-10 and IL-22: from Basic Science to Clinical Translation. Immunity 50, 871-891.
Peacock, J. W., Palmer, J., Fink, D., Ip, S., Pietras, E. M., Mui, A. L., Chung, S. W., Gleave, M. E., Cox, M. E., Parsons, R., Peter, M. E. & Ong, C. J. 2009. PTEN loss promotes mitochondrially dependent type II Fas-induced apoptosis via PEA-15. Mol Cell Biol 29, 1222-34.
Peters, A., Fowler, K. D., Chalmin, F., Merkler, D., Kuchroo, V. K. & Pot, C. 2015. IL-27 Induces Th17 Differentiation in the Absence of STAT1 Signaling. Journal of immunology 195, 4144-53.
Pauls, S. D. & Marshall, A. J. Regulation of immune cell signaling by SHIP1: A phosphatase, scaffold protein, and potential therapeutic target. Eur J Immunol 47, 932-945 (2017).
Philip A M, Wang Y, Mauro A, El-Rass S, Marshall J C, Lee W L, Slutsky A S, dosSantos C C, Wen X Y. (2017) Development of a zebrafish sepsis model for high-throughput drug discovery. Mol Med. 2017 Jun. 7; 23. PubMed PMID: 28598490.
Qiang, Y. W., Kopantzev, E. & Rudikoff, S. Insulinlike growth factor-I signaling in multiple myeloma: downstream elements, functional correlates, and pathway cross-talk. Blood 99, 4138-4146 (2002).
Qiao, Z., Xia, C., Shen, S., Corwin, F. D., Liu, M., Guan, R., Grider, J. R. & Qiao, L. Y. 2014. Suppression of the PI3K pathway in vivo reduces cystitis-induced bladder hypertrophy and restores bladder capacity examined by magnetic resonance imaging. PloS one 9, e114536.
Rittirsch D, Huber-Lang M S, Flierl M A, et al. Immunodesign of experimental sepsis by cecal ligation and puncture. Nature Protocols. 4, 31-36, 2009
Shi, Y., et al. Enhanced sensitivity of multiple myeloma cells containing PTEN mutations to CCI-779. Cancer research 62, 5027-5034 (2002).
Shouval, D. S., et al. Chapter Five—Interleukin 10 Receptor Signaling: Master Regulator of Intestinal Mucosal Homeostasis in Mice and Humans. in Advances in Immunology, Vol. 122 (ed. Alt, F. W.) 177-210 (Academic Press, 2014).
Steck, P. A., et al. Identification of a candidate tumour suppressor gene, MMAC1, at chromosome 10q23.3 that is mutated in multiple advanced cancers. Nat Genet 15, 356-362 (1997).
Stenton, G. R., Mackenzie, L. F., Tam, P., Cross, J. L., Harwig, C., Raymond, J., Toews, J., Chernoff, D., Macrury, T. & Szabo, C. 2013a. Characterization of AQX-1125, a small-molecule SHIP1 activator: Part 2. Efficacy studies in allergic and pulmonary inflammation models in vivo. British journal of pharmacology 168, 1519-29.
Stenton, G. R., Mackenzie, L. F., Tam, P., Cross, J. L., Harwig, C., Raymond, J., Toews, J., Wu, J., Ogden, N., Macrury, T. & Szabo, C. 2013b. Characterization of AQX-1125, a small-molecule SHIP1 activator: Part 1. Effects on inflammatory cell activation and chemotaxis in vitro and pharmacokinetic characterization in vivo. British journal of pharmacology 168, 1506-18.
Sydora, B. C., Tavernini, M. M., Wessler, A., Jewell, L. D. & Fedorak, R. N. 2003. Lack of interleukin-10 leads to intestinal inflammation, independent of the time at which luminal microbial colonization occurs. Inflamm Bowel Dis 9, 87-97.
Takeda, K., Clausen, B. E., Kaisho, T., Tsujimura, T., Terada, N., Forster, I. & Akira, S. 1999. Enhanced Th1 activity and development of chronic enterocolitis in mice devoid of Stat3 in macrophages and neutrophils. Immunity 10, 39-49.
Thakur P C, Davison J M, Stuckenholz C, Lu L, Bahary N. Dysregulated phosphatidylinositol signaling promotes endoplasmic-reticulum-stress-mediated intestinal mucosal injury and inflammation in zebrafish. Dis Model Mech. 2014 January; 7(1):93-106. doi: 10.1242/dmm.012864. Epub 2013 Oct. 17. PubMed PMID: 24135483; PubMed Central PMCID: PMC3882052.
Tu, Y., Gardner, A. & Lichtenstein, A. The phosphatidylinositol 3-kinase/AKT kinase pathway in multiple myeloma plasma cells: roles in cytokine-dependent survival and proliferative responses. Cancer research 60, 6763-6770 (2000).
Van Petegem, F., Clark, K. A., Chatelain, F. C. & Minor, D. L., JR. 2004. Structure of a complex between a voltage-gated calcium channel beta-subunit and an alpha-subunit domain. Nature 429, 671-5.
Vanderwinden, J. M., et al. Differences in signaling pathways and expression level of the phosphoinositide phosphatase SHIP1 between two oncogenic mutants of the receptor tyrosine kinase KIT. Cell Signal 18, 661-669 (2006).
Verstockt, B., Smith, K. G. & Lee, J. C. Genome-wide association studies in Crohn's disease: Past, present and future. Clin Transl Immunology 7, e1001 (2018).
Vivanco, I. & Sawyers, C. L. The phosphatidylinositol 3-Kinase AKT pathway in human cancer. Nat Rev Cancer 2, 489-501 (2002).
Wang S B, Deng Y Q, Ren J, Xiao B K, Liu Z, Tao Z Z. Exogenous interleukin-10 alleviates allergic inflammation but inhibits local interleukin-10 expression in a mouse allergic rhinitis model. BMC Immunol. 2014 Feb. 25; 15:9. doi: 10.1186/1471-2172-15-9. PubMed PMID: 24568666; PubMed Central PMCID: PMC3939634.
Yasukawa, H., Ohishi, M., Mori, H., Murakami, M., Chinen, T., Aki, D., Hanada, T., Takeda, K., Akira, S., Hoshijima, M., Hirano, T., Chien, K. R. & Yoshimura, A. 2003. IL-6 induces an anti-inflammatory response in the absence of SOCS3 in macrophages. Nat Immunol 4, 551-6.
Zhang, J., Choi, Y., Mavromatis, B., Lichtenstein, A. & Li, W. Preferential killing of PTEN-null myelomas by PI3K inhibitors through Akt pathway. Oncogene 22, 6289-6295 (2003).
Zhao S, Xia J, Wu X, Zhang L, Wang P, Wang H, Li H, Wang X, Chen Y, Agnetti J, Li Y, Pei D, Shu X. Deficiency in class III PI3-kinase confers postnatal lethality with IBD-like features in zebrafish. Nat Commun. 2018 Jul. 6; 9(1):2639. doi: 10.1038/s41467-018-05105-8. PubMed PMID: 29980668; PubMed Central PMCID: PMC6035235.
Zhou Y, Chen K, He L, Xia Y, Dai W, Wang F, Li J, Li S, Liu T, Zheng Y, Wang J, Lu W, Yin Q, Lu J, Teng H, Guo C. The Protective Effect of Resveratrol on Concanavalin-A-Induced Acute Hepatic Injury in Mice. Gastroenterol Res Pract. 2015; 2015:506390.
Zhu, J., Hou, T. & Mao, X. Discovery of selective phosphatidylinositol 3-kinase inhibitors to treat hematological malignancies. Drug Discovery Today 20, 988-994 (2015).
Zhu, J., Wang, M., Cao, B., Hou, T. & Mao, X. Targeting the Phosphatidylinositol 3-Kinase/AKT Pathway for the Treatment of Multiple Myeloma. Current Medicinal Chemistry 21, 3173-3187 (2014).
Zigmond, E., Bernshtein, B., Friedlander, G., Walker, Catherine R., Yona, S., Kim, K.-W., Brenner, O., Krauthgamer, R., Varol, C., Muller, W. & Jung, S. 2014. Macrophage-Restricted Interleukin-10 Receptor Deficiency, but Not IL-10 Deficiency, Causes Severe Spontaneous Colitis. Immunity 40, 720-733.

1. A compound of Formula I or a pharmaceutically acceptable salt, solvate, prodrug and/or derivative thereof 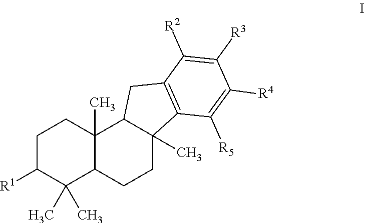
or a pharmaceutically acceptable salt, solvate, prodrug and/or derivative thereof,
wherein R¹ is selected from H, OH, OC₁₋₃alkyl, OC(O)C₁₋₃alkyl, NH₂, NHC₁₋₃alkyl, NHSO₂C₁₋₃alkyl, NSuccinamide, and NHC(O)C₁₋₃alkyl;
wherein R², R³, R⁴, and R⁵ are independently selected from H, OH, C₁₋₃alkyl, OC₁₋₃alkyl, NH₂, NHC₁₋₃alkyl, NHSO₂C₁₋₃alkyl, and NHC(O)C₁₋₃alkyl; or R² and R³, R³ and R⁴ or R⁴ and R⁵ taken together with the atoms they are attached to form a substituted or unsubstituted 5- or 6-membered heterocycle comprising at least one NH and optionally one or more additional heteroatoms selected from N, O, and S; and
wherein when R² and R³, R³ and R⁴ or R⁴ and R⁵ are taken together to form the substituted or unsubstituted 5- or 6-membered heterocycle, R⁴ and R⁵, R² and R⁵, or R² and R³ respectively are independently selected from H and C₁₋₃alkyl.

2. The compound of claim 1, wherein the compound of Formula I is a compound of Formula IA 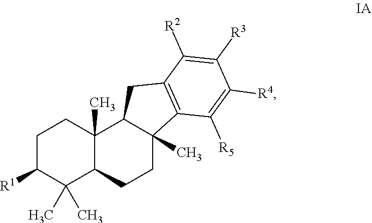
or an enantiomer thereof or a pharmaceutically acceptable salt, solvate, prodrug and/or derivative thereof.

3. The compound of claim 1, wherein R¹ is selected from H, NH₂, NHC₁₋₃alkyl, NHSO₂C₁₋₃alkyl, NSuccinamide, and NHC(O)C₁₋₃alkyl.

4. The compound of claim 1, wherein R² and R⁴ are H, and R³ and R⁵ are selected from OH, C₁₋₃alkyl, OC₁₋₃alkyl, NH₂, NHC₁₋₃alkyl, NHSO₂C₁₋₃alkyl, and NHC(O)C₁₋₃alkyl.

5. The compound of claim 4, wherein R³ and R⁵ are selected from OH, CH₃, OCH₃, NHSO₂CH₃, and NHC(O)CH₃.

6. The compound of claim 4, wherein R³ is selected from OH, OCH₃, NHSO₂CH₃, and NHC(O)CH₃; and R⁵ is CH₃.

7. The compound of claim 1, wherein R², R⁴, and R⁵ are H, and R³ is selected from OH, OC₁₋₃alkyl, NH₂, NHC₁₋₃alkyl, NHSO₂C₁₋₃alkyl, and NHC(O)C₁₋₃alkyl.

8. The compound of claim 7, wherein R³ is selected from OH, OCH₃, NHSO₂CH₃, and NHC(O)CH₃.

9. The compound of claim 1, wherein the substituted or unsubstituted 5- or 6-membered heterocycle are selected from

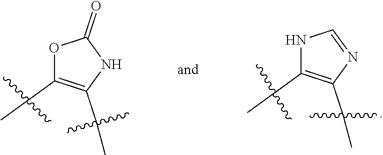 [SMILES: *c1[nH]c(=O)oc1* ; *c1nc[nH]c1*]

10. The compound of claim 1, wherein R² and R³ taken together with the atoms they are attached to form the substituted or unsubstituted 5- or 6-membered heterocycle, and R⁴ and R⁵ are independently selected from H and C₁₋₃alkyl.

11. The compound of claim 1, wherein the compound of Formula I is selected from 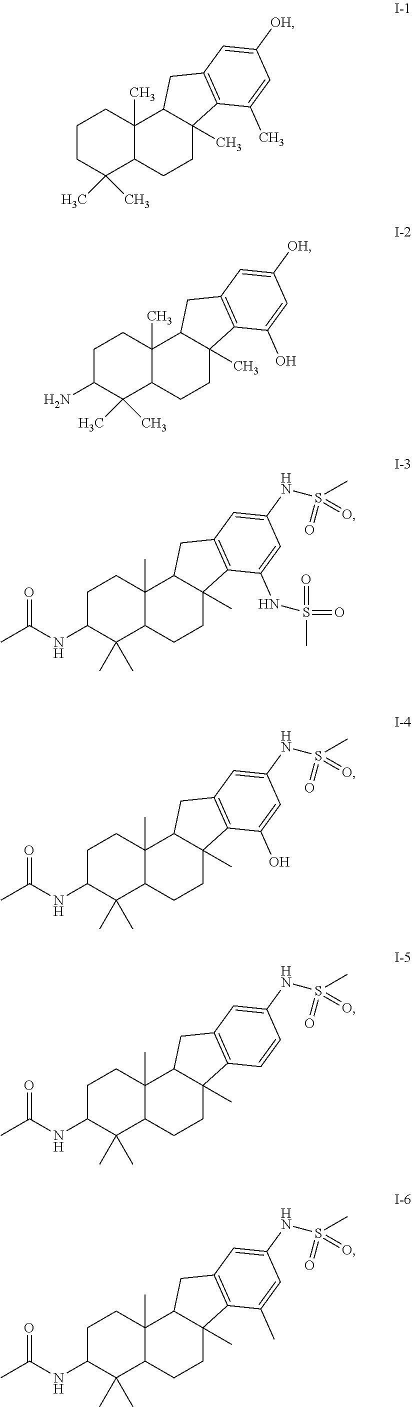 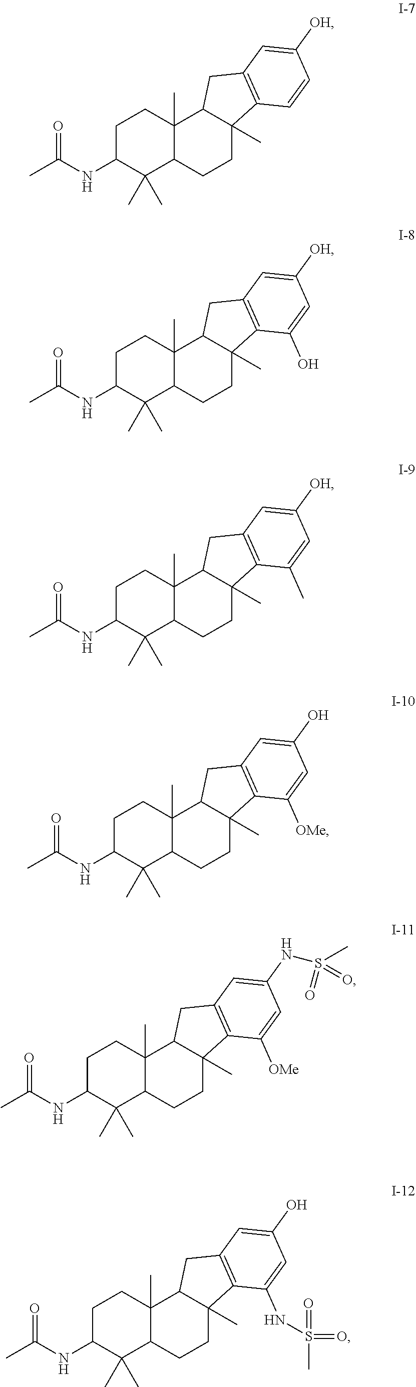 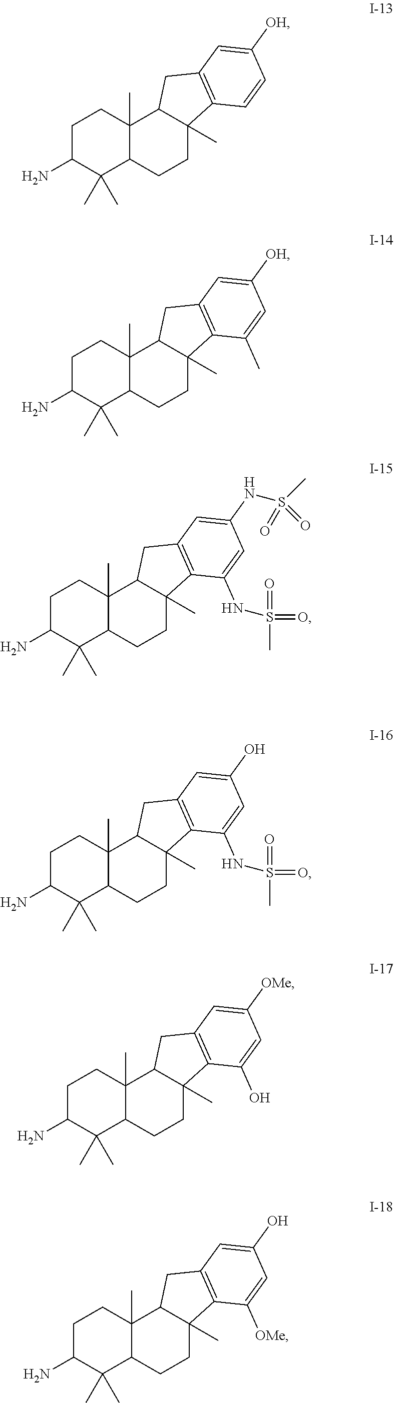 [SMILES: CC12CCC3C(C)(C)C(N)CCC3(C)C1Cc1cc(O)ccc12 ; Cc1cc(O)cc2c1C1(C)CCC3C(C)(C)C(N)CCC3(C)C1C2 ; CC12CCC3C(C)(C)C(N)CCC3(C)C1Cc1cc(NS(C)(=O)=O)cc(NS(C)(=O)=O)c12 ; CC12CCC3C(C)(C)C(N)CCC3(C)C1Cc1cc(O)cc(NS(C)(=O)=O)c12 ; COc1cc(O)c2c(c1)CC1C2(C)CCC2C(C)(C)C(N)CCC12C ; COc1cc(O)cc2c1C1(C)CCC3C(C)(C)C(N)CCC3(C)C1C2] 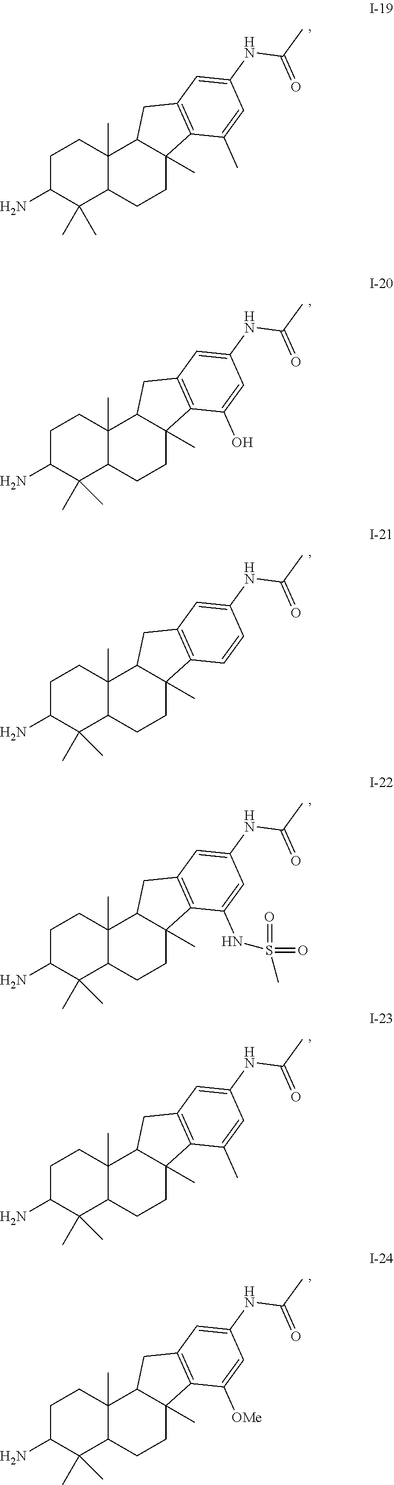 [SMILES: CC(=O)Nc1cc(C)c2c(c1)CC1C2(C)CCC2C(C)(C)C(N)CCC12C ; CC(=O)Nc1cc(O)c2c(c1)CC1C2(C)CCC2C(C)(C)C(N)CCC12C ; CC(=O)Nc1ccc2c(c1)CC1C2(C)CCC2C(C)(C)C(N)CCC12C ; CC(=O)Nc1cc2c(c(NS(C)(=O)=O)c1)C1(C)CCC3C(C)(C)C(N)CCC3(C)C1C2 ; CC(=O)Nc1cc(C)c2c(c1)CC1C2(C)CCC2C(C)(C)C(N)CCC12C ; COc1cc(NC(C)=O)cc2c1C1(C)CCC3C(C)(C)C(N)CCC3(C)C1C2] 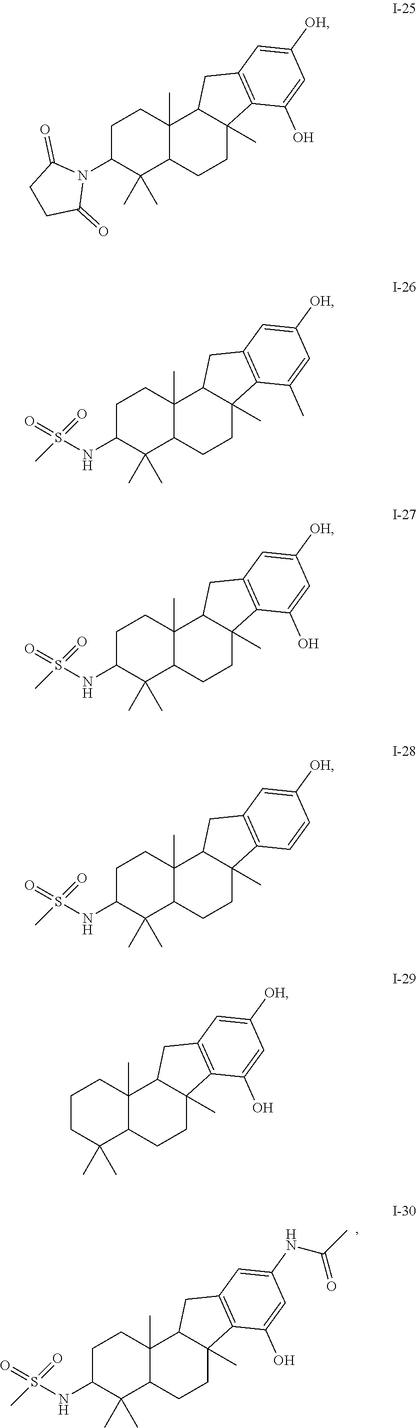 [SMILES: CC12CCC3C(C)(C)C(N4C(=O)CCC4=O)CCC3(C)C1Cc1cc(O)cc(O)c12 ; Cc1cc(O)cc2c1C1(C)CCC3C(C)(C)C(NS(C)(=O)=O)CCC3(C)C1C2 ; CC12CCC3C(C)(C)C(NS(C)(=O)=O)CCC3(C)C1Cc1cc(O)cc(O)c12 ; CC12CCC3C(C)(C)C(NS(C)(=O)=O)CCC3(C)C1Cc1cc(O)ccc12 ; CC1(C)CCCC2(C)C1CCC1(C)c3c(O)cc(O)cc3CC12 ; CC(=O)Nc1cc(O)c2c(c1)CC1C2(C)CCC2C(C)(C)C(NS(C)(=O)=O)CCC12C] 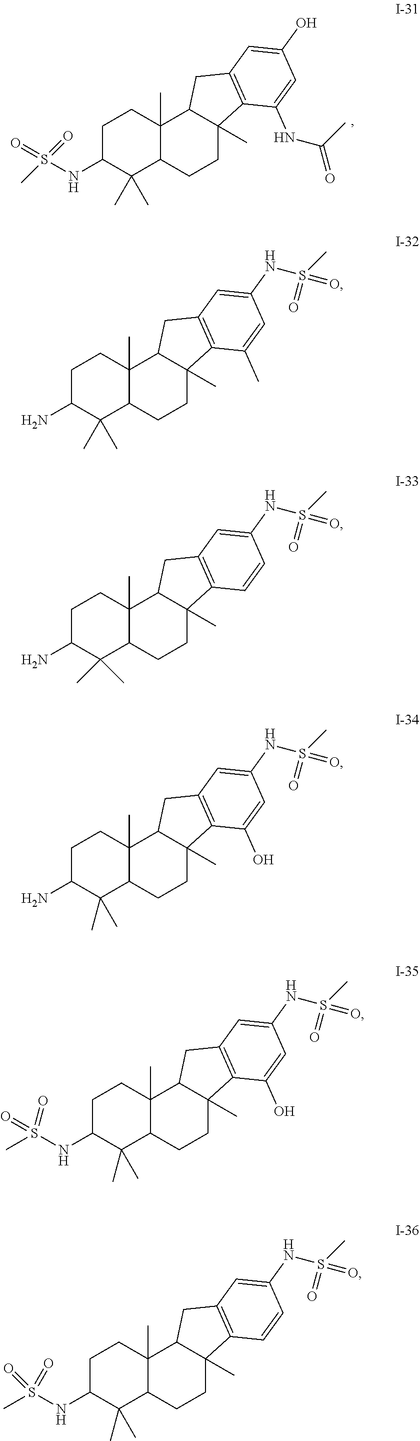 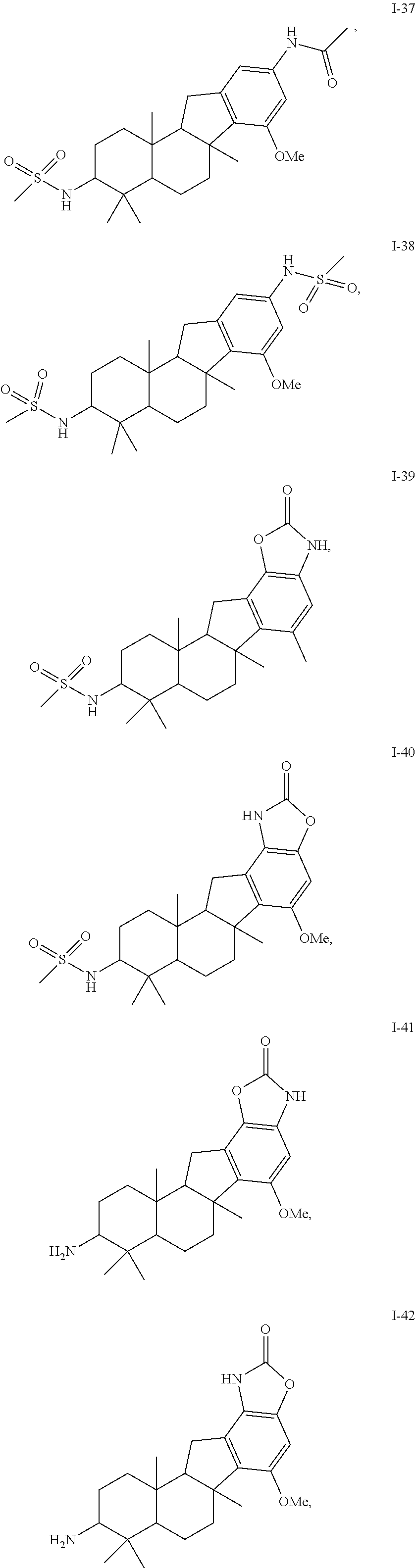 [SMILES: COc1cc(NC(C)=O)cc2c1C1(C)CCC3C(C)(C)C(NS(C)(=O)=O)CCC3(C)C1C2 ; COc1cc(NS(C)(=O)=O)cc2c1C1(C)CCC3C(C)(C)C(NS(C)(=O)=O)CCC3(C)C1C2 ; Cc1cc2[nH]c(=O)oc2c2c1C1(C)CCC3C(C)(C)C(NS(C)(=O)=O)CCC3(C)C1C2 ; COc1cc2oc(=O)[nH]c2c2c1C1(C)CCC3C(C)(C)C(NS(C)(=O)=O)CCC3(C)C1C2 ; COc1cc2[nH]c(=O)oc2c2c1C1(C)CCC3C(C)(C)C(N)CCC3(C)C1C2 ; COc1cc2oc(=O)[nH]c2c2c1C1(C)CCC3C(C)(C)C(N)CCC3(C)C1C2] 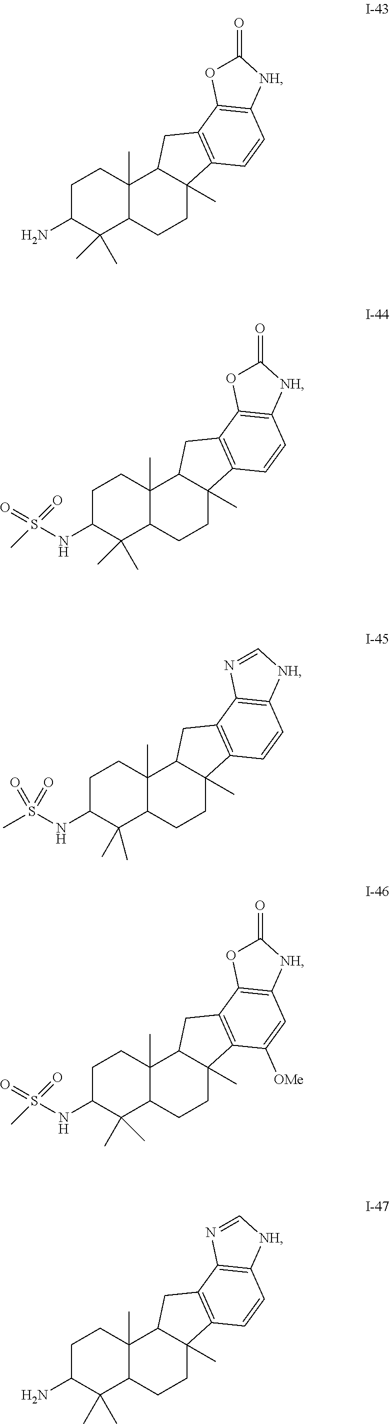 [SMILES: CC12CCC3C(C)(C)C(N)CCC3(C)C1Cc1c2ccc2[nH]c(=O)oc12 ; CC12CCC3C(C)(C)C(NS(C)(=O)=O)CCC3(C)C1Cc1c2ccc2[nH]c(=O)oc12 ; CC12CCC3C(C)(C)C(NS(C)(=O)=O)CCC3(C)C1Cc1c2ccc2[nH]cnc12 ; COc1cc2[nH]c(=O)oc2c2c1C1(C)CCC3C(C)(C)C(NS(C)(=O)=O)CCC3(C)C1C2 ; CC12CCC3C(C)(C)C(N)CCC3(C)C1Cc1c2ccc2[nH]cnc12] 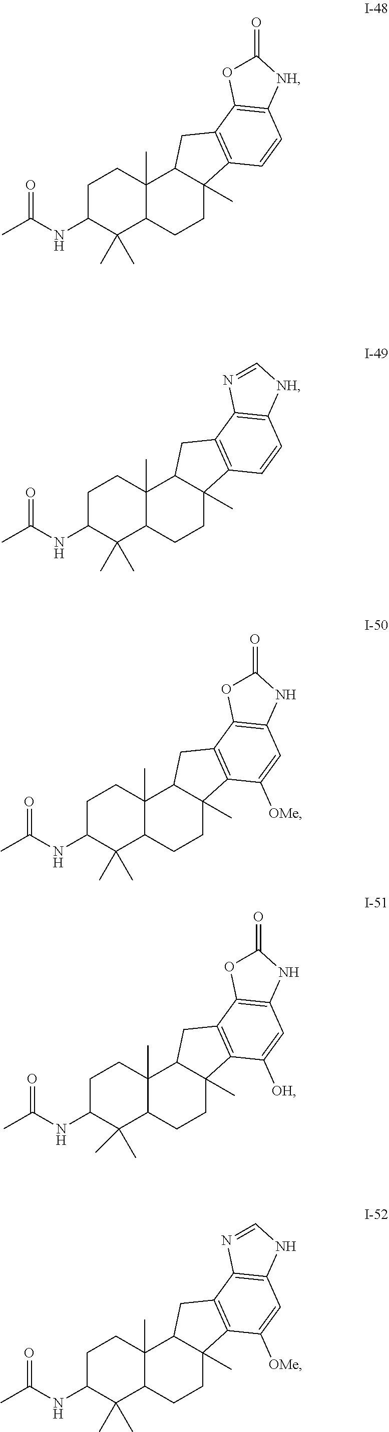 [SMILES: CC(=O)NC1CCC2(C)C3Cc4c(ccc5[nH]c(=O)oc45)C3(C)CCC2C1(C)C ; CC(=O)NC1CCC2(C)C3Cc4c(ccc5[nH]cnc45)C3(C)CCC2C1(C)C ; COc1cc2[nH]c(=O)oc2c2c1C1(C)CCC3C(C)(C)C(NC(C)=O)CCC3(C)C1C2 ; CC(=O)NC1CCC2(C)C3Cc4c(c(O)cc5[nH]c(=O)oc45)C3(C)CCC2C1(C)C ; COc1cc2[nH]cnc2c2c1C1(C)CCC3C(C)(C)C(NC(C)=O)CCC3(C)C1C2] 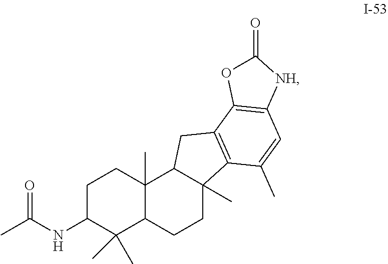 [SMILES: CC(=O)NC1CCC2(C)C3Cc4c(c(C)cc5[nH]c(=O)oc45)C3(C)CCC2C1(C)C]
and a pharmaceutically acceptable salt, solvate, prodrug and/or derivative thereof.

12. A method of treating a disease, disorder or condition mediated or treatable by activation of SHIP1 comprising administering a therapeutically effective amount of a compound of Formula I or a pharmaceutically acceptable salt, solvate, prodrug and/or derivative thereof as defined in claim 1 to a subject in need thereof.

13. The method of claim 12, wherein the compound of Formula I or the pharmaceutically acceptable salt, solvate, prodrug and/or derivative thereof is formulated in a composition comprising the compound of Formula I or the pharmaceutically acceptable salt, solvate, prodrug and/or derivative thereof and a pharmaceutically acceptable carrier.

14. The method of claim 12, wherein the disease, disorder or condition mediated or treatable by activation of SHIP1 is selected from inflammatory bowel disease (IBD), multiple myeloma, allergy, a neoplastic disorder such as colon cancer, sepsis, organ injury, trauma, cardiovascular diseases, osteoporosis and sleep disorders.

15. The method of claim 14, wherein the IBD is selected from Crohn's disease, and ulcerative colitis.

16. The method of claim 14, wherein the organ injury is liver injury, optionally the liver injury is selected from viral hepatitis, autoimmune hepatitis, primary biliary cirrhosis, and liver allograft rejection.

17. The method of claim 14, wherein the disease, disorder or condition mediated or treatable by activation of SHIP1 is multiple myeloma.

18. The method of claim 14, wherein the sepsis is severe sepsis, optionally severe sepsis caused by COVID-19.

19-20. (canceled)

21. The method of claim 12, wherein the compound for Formula I or the pharmaceutically acceptable salt, solvate, prodrug and/or derivative thereof is formulated in a composition comprising the compound for Formula I or the pharmaceutically acceptable salt, solvate, prodrug and/or derivative thereof, and a pharmaceutically acceptable carrier.

22. The method of claim 12, wherein the subject is human.

23-26. (canceled)
